(19) United States
(12) Patent Application Publication
(10) Pub. No.: US 2002/0094962 A1
(43) Pub. Date: Jul. 18, 2002

(54) Motilide compounds

(76) Inventors: Gary Ashley, Alameda, CA (US); Brian Metcalf, Moraga, CA (US); Zong-Qiang Tian, Fremont, CA (US); Daniel Santi, San Francisco, CA (US)

Correspondence Address: Carolyn A. Favorito, Morrison & Foerster LLP, Suite 500, 3811 Valley Centre Drive, San Diego, CA, 92130-2332, US

(21) Appl. No.: 09/990,554

(22) Filed: Nov. 21, 2001

Related U.S. Application Data
(60) Provisional application No. 60/250,640, filed on Dec. 1, 2000.
(60) Provisional application No. 60/269,632, filed on Feb. 15, 2001.

(51) Int. Cl. A61K 31/7048; C07H 17/08

(52) U.S. Cl. 514/28; 536/7.4; 536/17.4

(57) ABSTRACT

The present invention provides novel macrolide compounds of the formulas

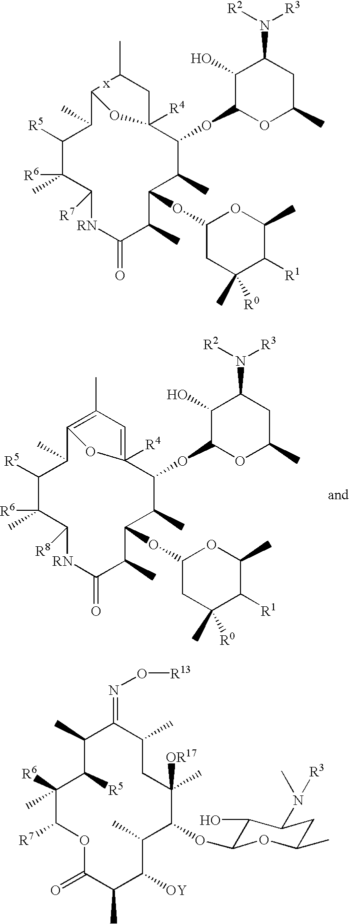

wherein:

R is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;

R⁰ is hydroxyl or methoxy;

R¹ is selected from the group consisting of hydrogen, hydroxyl, halide, NH₂, OR⁹,

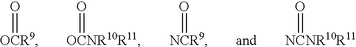

where

R⁹ is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl, and R¹⁰ and R¹¹ are each independently hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;

R¹ and R³ are each independently selected from the group consisting of hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl, or R² and R³ together form a cycloalkyl or an aryl moiety;

R⁴ is hydrogen or methyl;

R⁵ is hydroxyl or oxo;

R is hydrogen, hydroxyl, or OR¹² where R¹² is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, or unsubstituted C₂-C₁₀ alkynyl;

R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;

R⁸ is unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;

R¹³ is hydrogen, unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;

R¹⁷ is hydrogen or methyl;

x is a single or a double bond; and,

Y is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, unsubstituted alkynylaryl, unsubstituted cladinose, or substituted cladinose.

This application claims priority to U.S. Provisional Application No. 60/250,640 filed Dec. 1, 2000 entitled MACROLIDE COMPOUNDS by inventors Gary Ashley, Brian Metcalf, and Zong-Qiang Tian and U.S. Provisional Application No. 60/269,632 filed Feb. 15, 2001 entitled MACROLIDE COMPOUNDS by inventors Brian Metcalf and Daniel Santi, both of which are incorporated herein by reference.

### BACKGROUND

The present invention provides novel prokinetic agents with superior pharmacological and pharmacokinetic properties for the treatment of gastrointestinal motility disorders. The invention relates to the fields of chemistry, medicinal chemistry, medicine, molecular biology, and pharmacology.

Gastrointestinal (“GI”) motility regulates the orderly movement of ingested material through the gut to insure adequate absorption of nutrients, electrolytes and fluids. Appropriate transit through the esophagus, stomach, small intestine and colon depends on regional control of intraluminal pressure and several sphincters that regulate forward movement and prevent back-flow of GI contents. The normal GI motility pattern may be impaired by a variety of circumstances including disease and surgery.

Disorders of gastrointestinal motility include, for example, gastroparesis and gastroesophageal reflux disease (“GERD”). Gastroparesis is the delayed emptying of stomach contents. Symptoms of gastroparesis include stomach upset, heartburn, nausea, and vomiting. Acute gastroparesis may be caused by, for example, drugs (e.g., opiates), viral enteritis, and hyperglycemia, and is usually managed by treating the underlying disease rather than the motility disorder. The most common causes of chronic gastroparesis are associated with long standing diabetes or idiopathic pseudo-obstruction, often with so-called “non-ulcer” or “functional” dyspepsia.

GERD refers to the varied clinical manifestations of reflux of stomach and duodenal contents into the esophagus. The most common symptoms are heartburn and dysphasia; blood loss may also occur from esophageal erosion. GERD may be associated with low tone and inappropriate relaxation of the lower esophageal sphincter and occurs with gastroparesis in about 40% of cases. In most cases, GERD appears to be treatable with agents that reduce the release of acidic irritant by the stomach (e.g., Prilosec) or agents that increase the tone of the lower esophageal sphincter (e.g., cisapride). Other examples of disorders whose symptoms include impaired gastrointestinal motility are anorexia, gall bladder stasis, postoperative paralytic ileus, scleroderma, intestinal pseudoobstruction, gastritis, emesis, and chronic constipation (colonic inertia).

These GI disorders are generally treated with prokinetic agents that enhance propulsive motility. Motilides are macrolide compounds such as erythromycin and its derivatives that are agonists of the motilin receptor. Evidence of the potential clinical utility of motilides includes their ability to induce phase III of Migrating Motor Complexes (“MMC”). MMC refers to the four phases (I-IV) of electrical activity displayed by the stomach and small intestine in the fasting state. Muscular contraction occurs in phases III and IV which coincide with a peristaltic wave that propels enteric contents distally during fasting. Other clinically relevant effects include: increase in esophageal peristalsis and LES pressure in normal volunteers and patients with GERD; acceleration of gastric emptying in patients with gastric paresis; and stimulation of gallbladder contractions in normal volunteers, patients after gallstone removal, and diabetics with autonomic neuropathy.

The discovery of the first motilide compound was serendipitous. Since the 1950's, erythromycin A 1 has been known to cause GI side effects such as nausea, vomiting, and abdominal discomfort. These effects are now largely explained by the motilin agonist activity of erythromycin A and an acid catalyzed degradation product, 8,9-anhydro-6,9-hemiacetal 2, which is also known as the enol ether form.

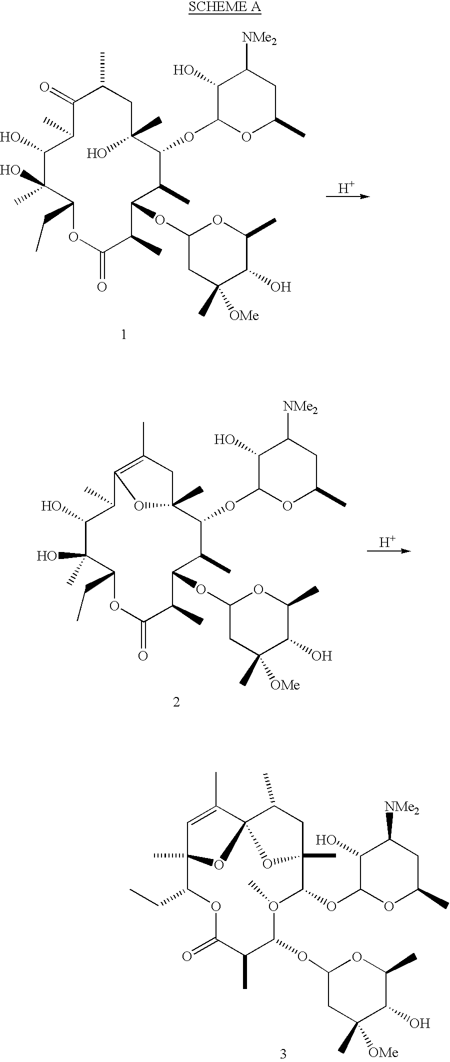

As illustrated by Scheme A, erythromycin A 1 undergoes an acid catalyzed rearrangement in the stomach to form the enol ether 2 which is then further degraded into the spiroketal 3. Both erythromycin A and the enol ether are motilin agonists but the spiroketal is not. Because the enol ether is approximately ten fold more potent as a motilin agonist than erythromycin A and does not also posses antimicrobial activity, the potential clinical uses of enol ether derivatives as prokinetic agents are being investigated.

Enol ether erythromycin derivatives under clinical investigation include EM-523 (4); EM -574 (5); LY267,108 (6); GM-611 (7); and ABT-229 (8) whose structures are shown below. See U.S. Pat. Nos. 5,578,579; 5,658,888; 5,922,849; 6,077,943; and 6,084,079 which are all incorporated herein by reference.

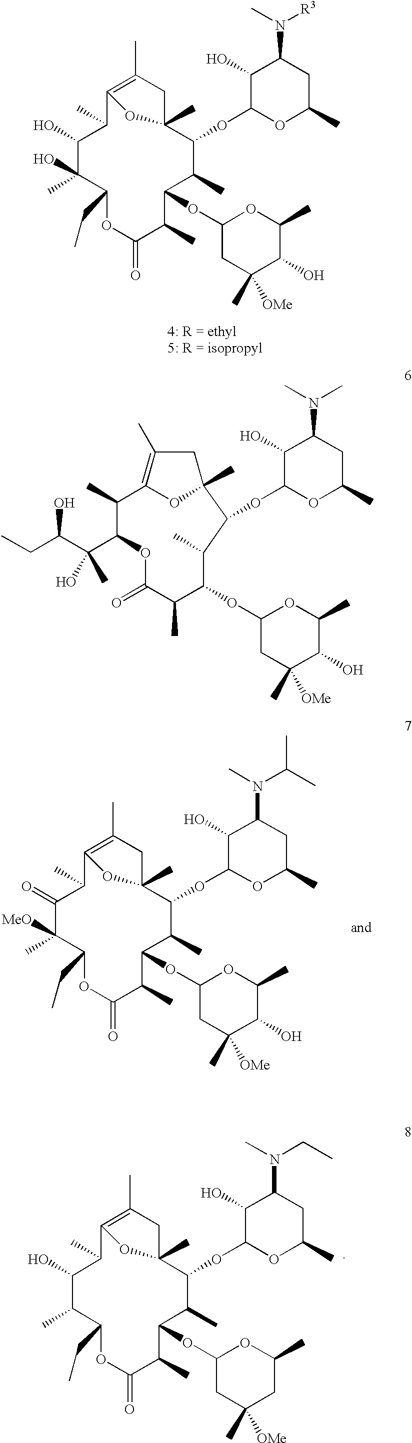

Other motilides of potential interest include lactam enol ethers and lactam epoxide derivatives. Illustrative examples of these lactam compounds include A-81648 (9) and A-173508 (10) whose structures are shown below. See also U.S. Pat. Nos. 5,712,253; 5,523,401; 5,523,418; 5,538,961; 5,554,605 which are incorporated herein by reference.

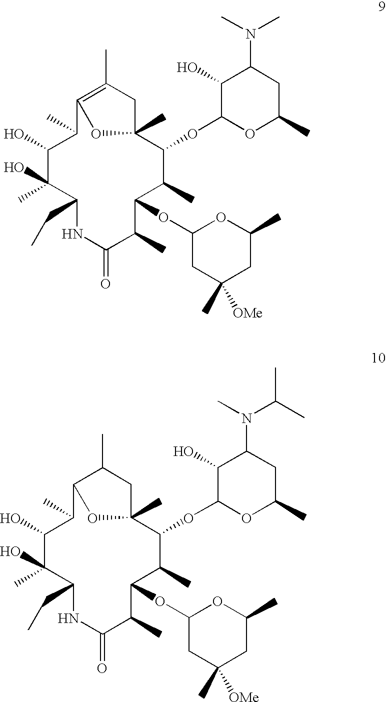

In general, these and other previously disclosed macrolides are synthetically accessible compounds that are derived from erythromycin A or B. Because nature has not optimized the erythromycin structure for its prokinetic activity, it is likely that the potency of motilide agonists could be greatly enhanced. Compounds resulting from such efforts could be of significant benefit in the treatment of wide variety of diseases and conditions. The present invention provides such compounds.

### DESCRIPTION OF THE PREFERRED EMBODIMENTS

The present invention provides novel macrolide compounds (or intermediates thereto) with superior pharmacological and pharmacokinetic properties for the treatment of gastrointestinal disorders where enhanced GI motiliy is indicated or desired. The compounds of the present invention typically are derived from “unnatural” erythromycins and generally differ from naturally occurring erythromycins A, B, C, and D by having a non-ethyl group (a group that is not —CH₂CH₃) or a substituted ethyl at C-13 and/or by having a hydrogen instead of a methyl group at C-6 (C-6 desmethyl compounds).

Definitions

Many of the inventive compounds contain one or more chiral centers. Unless indicated otherwise, all of the stereoisomers of a depicted structure are included within the scope of the invention, as pure compounds as well as mixtures of stereoisomers. Similarly, all geometric isomers are also included within the scope of the invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Listed below are definitions of various terms used to describe this invention. These definitions apply to the terms as they are used throughout this specification, unless otherwise limited in specific instances, either individually or as part of a larger group.

The term “alkyl” or “unsubstituted alkyl” refers to a straight, branched or cyclic hydrocarbon. “Alkenyl” or “un substituted alkenyl” refers to a straight, branched, or cyclic chain hydrocarbon with at least one carbon-carbon double bond. “Alkynyl” or “unsubstituted alkynyl” refers to a straight, branched, or cyclic hydrocarbon with at least one carbon-carbon triple bound. Substituted alkyl, substituted alkenyl, or substituted alkynyl refer to the respective alkyl, alkenyl or alkynyl group substituted by one or more substituents. Illustrative examples of substituents include but are not limited to alkyl, alkenyl, alkynyl, aryl, halo; trifluoromethyl; trifluoromethoxy; hydroxy; alkoxy; cycloalkoxy; heterocyclooxy; oxo (═O); alkanoyl (—C(═O)-alkyl); aryloxy; alkanoyloxy; amino; alkylamino; arylamino; aralkylamino; cycloalkylamino; heterocycloamino; disubstituted amines in which the two amino substituents are selected from alkyl, aryl, or aralkyl; alkanoylamino; aroylamino; aralkanoylamino; substituted alkanoylamino; substituted arylamino; substituted aralkanoylamino; thiol; alkylthio; arylthio; aralkylthio; cycloalkylthio; heterocyclothio; alkylthiono; arylthiono; aralkylthiono; alkylsulfonyl; arylsulfonyl; aralkylsulfonyl; sulfonamido (e.g., SO₂NH₂); substituted sulfonamido; nitro; cyano; carboxy; carbamyl (e.g., CONH₂); substituted carbamyl (e.g., —C(═O)NR′R″ where R′ and R″ are each independently hydrogen, alkyl, aryl, aralkyl and the like); alkoxycarbonyl, aryl, guanidino, and heterocyclo such as indoyl, imidazolyl, furyl, thienyl, thiazolyl, pyrrolidyl, pyridyl, pyrimidyl and the like. Where applicable, the substituent may be further substituted such as with halogen, alkyl, alkoxy, aryl, or aralkyl and the like.

The term “aryl” or “unsubstituted aryl” refers to an aromatic ring having 6 to 12 carbon atoms and includes heteroaryls (aryls that have one or more heteroatoms such as N, S and O). Illustrative examples of aryl include but are not limited to biphenyl, furyl, imidazolyl, indolyl, isoquinolyl, naphthyl, oxazolyl, phenyl, pyridyl, pyrryl, quinolyl, quinoxalyl, tetrazoyl, thiazoyl, thienyl and the like. Substituted aryl refers to an aryl group substituted by, for example, one to four substituents such as substituted and unsubstituted alkyl, alkenyl, alkynyl, and aryl; halo; trifluoromethoxy; trifluoromethyl; hydroxy; alkoxy; cycloalkyloxy; heterocyclooxy; alkanoyl; alkanoyloxy; amino; alkylamino; aralkylamino; cycloalkylamino; heterocycloamino; dialkylamino; alkanoylamino; thio; alkylthio; cycloalkylthio; heterocyclothio; ureido; nitro; cyano; carboxy; carboxyalkyl; carbamyl; alkoxycarbonyl; alkylthiono; arylthiono; alkylsulfonyl; sulfonamido; aryloxy; and the like. The substituent may be further substituted, for example, by halo, hydroxy; alkyl, alkoxy; aryl, substituted aryl, substituted alkyl, substituted aralkyl, and the like.

The terms “alkylaryl” or “arylalkyl” (or “unsubstituted alkylaryl or ” unsubstituted arylalkyl) refer to an aryl group bonded directly through an alkyl group, such as benzyl. Similarly, “alkenylaryl” and “arylalkenyl” (or “unsubstituted alkenylaryl” or “unsubstituted arylalkenyl”) refer to an aryl group bonded directly through an alkenyl group and “alkynylaryl” and “arylalkynyl” (or “unsubstituted alkynylaryl or ” unsubstituted arylalkenyl”) refer to an aryl group bonded directly through an alkynyl group. Substituted counterparts of these moieties are the respective moiety that is substituted by one or more substituents.

The term amidoalkylaryl refer to a group of the formula —ZNH—(C═O)—R′R″ where Z may be present or absent, and Z and R′ re each independently a substituted or unsubstituted C₁-C₁₀ alkyl, alkenyl, or alkynyl and R″ is a substituted or unsubstituted aryl.

The terms “halogen,” “halo”, or “halide” refer to fluorine, chlorine, bromine and iodine.

The term “erythromycin” refers to a compound of the formula

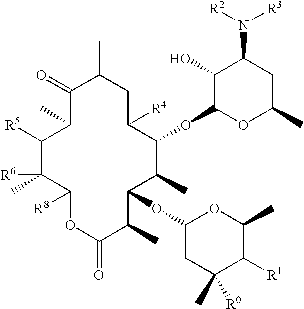

where R⁰, R¹, R², R³, R⁴, R⁵, R⁶, and R⁸ are as described herein and derivatives and analogs thereof.

Free hydroxyl groups in the compounds of the present invention may optionally be protected with a hydroxyl protecting group. The term “hydroxy protecting group” refers to groups known in the art for such purpose. Commonly used hydroxy protecting groups are disclosed, for example, in T. H. Greene and P. G. M. Wuts, Protective Groups in Organic Synthesis, 2^{nd} edition, John Wiley & Sons, New York (1991), which is incorporated herein by reference. Illustrative hydroxyl protecting groups include but not limited to tetrahydropyranyl; benzyl; methylthiomethyl; ethythiomethyl; pivaloyl; phenylsulfonyl; triphenylmethyl; trisubstituted silyl such as trimethyl silyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl and the like; acyl and aroyl such as acetyl, pivaloylbenzoyl, 4-methoxybenzoyl, 4-nitrobenzoyl and aliphatic acylaryl and the like. Hydroxyl protected versions of the inventive compounds are also encompassed within the scope of the present invention.

In addition to the explicit substitutions at the above-described groups, the inventive compounds may include other substitutions where applicable. For example, the erythromycin backbone or backbone substituents may be additionally substituted (e.g., by replacing one of the hydrogens or by derivatizing a non-hydrogen group) with one or more substituents such as C₁-C₅ alkyl, C₁-C₅ alkoxy, phenyl, or a functional group. Illustrative examples of suitable functional groups include but are not limited to alcohol, sulfonic acid, phosphine, phosphonate, phosphonic acid, thiol, ketone, aldehyde, ester, ether, amine, quaternary ammonium, imine, amide, imide, imido, nitro, carboxylic acid, disulfide, carbonate, isocyanate, carbodiimide, carboalkoxy, carbamate, acetal, ketal, boronate, cyanohydrin, hydrazone, oxime, hydrazide, enamine, sulfone, sulfide, sulfenyl, and halogen.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

Compounds of the Present Invention

In one aspect of the present invention, compounds are provided having the structure

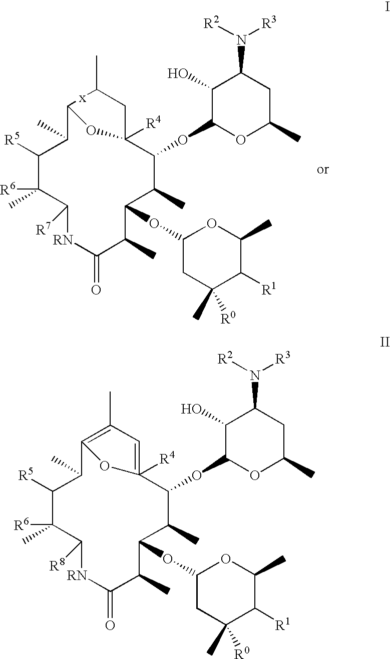

wherein:

R is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;

R⁰ is hydroxyl or methoxy;

R¹ is selected from the group consisting of hydrogen, hydroxyl, halide, NH_{2,} OR⁹,

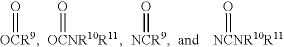

where

R⁹ is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl, and R¹⁰ and R¹¹ are each independently hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;

R² and R³ are each independently selected from the group consisting of hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C10 alkynyl, substituted aryl, unsubstituted aryl, substituted alkylaryl, unsubstituted alkylaryl, substituted alkenylaryl, unsubstituted alkenylaryl, substituted alkynylaryl, and unsubstituted alkynylaryl, or R² and R³ together form a cycloalkyl or an aryl moiety;

R⁴ is hydrogen or methyl;

R⁵ is hydroxyl or oxo;

R⁶ is hydrogen, hydroxyl or OR¹² where R¹² is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, or unsubstituted C₂-C₁₀ alkynyl;

R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;

R⁸ is unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl; and,

x is a single or a double bond.

In another aspect of the present invention, compounds are provided of structure I wherein the C-8 carbon is in the R configuration. In another aspect of the present invention, compounds are provided of structure I wherein the C-9 carbon is in the R configuration. In another aspect of the present invention, compounds are provided of structure I wherein the C-8 and C-9 carbons are both in the R configuration.

In another aspect of the present invention, compounds are provided of structures I and II wherein: R is hydrogen, substituted C₁-C₅ alkyl, unsubstituted C₁-C₅ alkyl, substituted aryl, unsubstituted aryl, substituted alkylaryl, or unsubstituted alkylaryl; R⁰ is hydroxyl or methoxy; R¹ is hydrogen or hydroxyl; R² and R³ are each independently substituted C¹-C₅ alkyl, unsubstituted C₁-C₅ alkyl, substituted phenyl, unsubstituted phenyl, substituted benzyl or unsubstituted benzyl; R⁴ is methyl; R⁵ is hydroxyl or oxo; R⁶ is hydrogen, hydroxyl or OR¹² wherein R¹² is substituted C₁-C₅ alkyl or unsubstituted C₁-C₅ alkyl; R⁷ is substituted methyl, unsubstituted methyl, substituted C₃-C₅ alkyl, unsubstituted C₃-C₅ alkyl, substituted C₂-C₅ alkenyl, unsubstituted C₂-C₅ alkenyl, substituted C₂-C₅ alkynyl, unsubstituted C₂ -C₅ alkynyl, substituted aryl, unsubstituted aryl, substituted alkylaryl or alkenylaryl; R⁸ is substituted C₁-C₅ alkyl, unsubstituted C₁-C₅ alkyl, substituted C₂-C₅ alkenyl, unsubstituted C₂-C₅ alkenyl, substituted C₂-C₅ alkynyl, unsubstituted C₂-C₅ alkynyl, substituted aryl, unsubstituted aryl, substituted alkylaryl, unsubstituted alkylaryl, substituted alkenylaryl, unsubstituted alkenylaryl; and, x is single bond or a double bond.

In another aspect of the present invention, compounds are provided of structures I and II wherein: R is hydrogen, C₁-C₅ alkyl, aryl, or alkylaryl; R⁰ is hydroxyl or methoxy; R¹ is hydrogen or hydroxyl; R² and R³ are each independently C₁-C₅ alkyl, phenyl or benzyl; R⁴ is methyl; R⁵ is hydroxyl or oxo; R⁶ is hydrogen, hydroxyl or methoxy; R⁷ and R⁸ are amidoalkylaryl.

In another aspect of the present invention, compounds are provided of structures I and II wherein: R is hydrogen, methyl, ethyl, propyl, isopropyl, phenyl or benzyl; R⁰ is hydroxyl or methoxy; R¹ is hydrogen or hydroxyl; R² is methyl; R³ is methyl, ethyl, propyl, isopropyl, butyl, isobutyl, secbutyl or tertbutyl; R⁴ is methyl; R⁵ is hydroxyl; R⁶ is hydroxyl or methoxy; R⁷ is methyl, vinyl, propyl, isobutyl, pentyl, prop-2-enyl, propargyl, but-3-enyl, 2-azidoethyl, 2-fluoroethyl, 2-chloroethyl, cyclohexyl, phenyl, or benzyl; R⁸ is methyl, ethyl vinyl, propyl, isobutyl, pentyl, prop-2-enyl, propargyl, but-3-enyl, 2-azidoethyl, 2-fluoroethyl, 2-chloroethyl, cyclohexyl, phenyl, or benzyl; and, x is a single or a double bond.

In another aspect of the present invention, compounds are provided of structures I and II wherein: R is hydrogen; R⁰ is methoxy; R¹ is hydrogen or hydroxyl; R² is methyl; R³ is methyl, ethyl, or isopropyl; R⁴ is methyl; R⁵ is hydroxyl; R⁶ is hydroxyl; R⁷ is propyl, but-3-enyl, 2-azidoethyl, phenyl, or benzyl; R⁸ is ethyl, propyl, but-3-enyl, 2-azidoethyl, phenyl, or benzyl; and, x is a single or a double bond.

In another aspect of the present invention, compounds of the following structures are provided

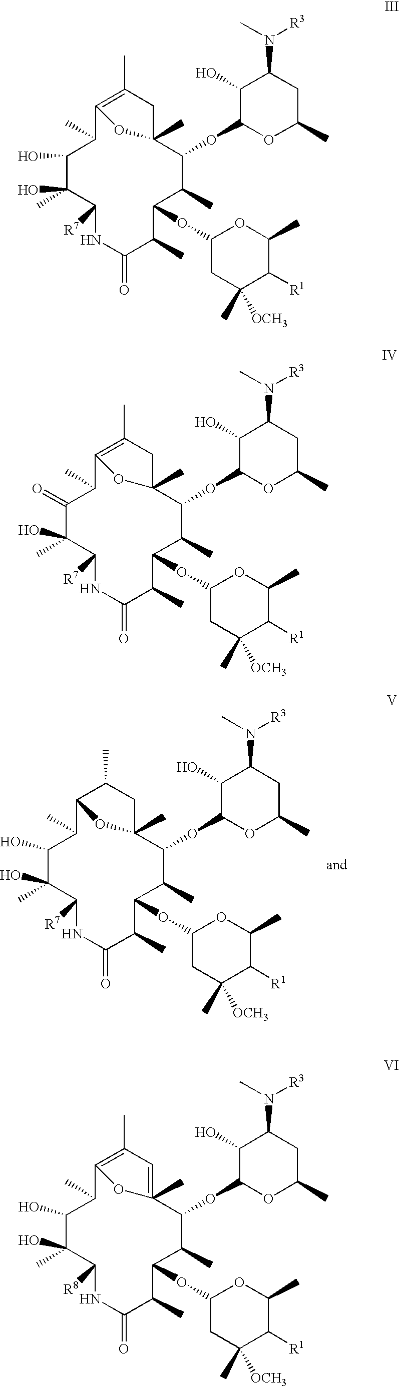

wherein R¹ is hydrogen or hydroxyl; R³ is methyl, ethyl, or isopropyl; R⁷ is propyl or fluoroethyl; and R⁸ is ethyl, fluoroethyl, or propyl.

In another aspect of the present invention, compounds are provided having the structure

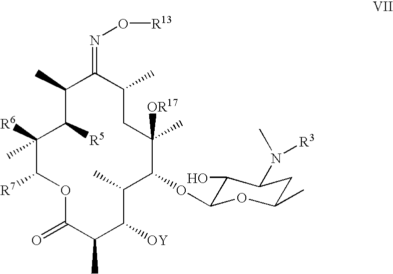

Y is hydrogen, substituted C₁-C₁₀ alkyl, unsubstituted C₁-C₁₀ alkyl, substituted C2-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, unsubstituted alkynylaryl, unsubstituted cladinose, or substituted cladinose;

R³ is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;

R⁵ is hydroxyl or oxo;

R⁶ is hydrogen, hydroxyl or OR¹² where R¹² is substituted C₁-C₁₀ alkyl, unsubstituted C₁-C₁₀ alkyl, substituted C₂-C₁₀ alkenyl, unsubstituted C₂-C10 alkenyl, substituted C₂-C₁₀ alkynyl, or unsubstituted C₂-C₁₀ alkynyl;

R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;

R¹³ is hydrogen, unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl; and,

R¹⁷ is hydrogen or methyl.

In another aspect of the present invention, compounds are provided of structure VII wherein

R³ is substituted C₃-C₁₀ alkyl, unsubstituted C₃-C₁₀ alkyl, substituted C₄-C₁₀ alkenyl, unsubstituted C₄-C₁₀ alkenyl, substituted aryl, unsubstituted aryl, substituted alkylaryl, unsubstituted alkylaryl, substituted alkenylaryl, or unsubstituted alkenylaryl;

R⁵ and R⁶ are both hydroxyl;

R⁷ is propyl or fluoroethyl;

R¹⁷ is hydrogen or methyl; and

Y is cladinose, 4-acyl-cladinose, 4-sulfonyl-cladinose, or 4-carbamoyl-cladinose.

In another aspect of the present invention, compounds are provided having the structure

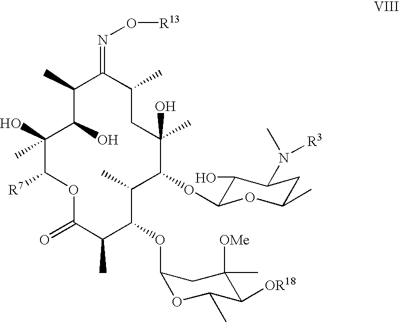

R³ is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;

R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;

R13 is hydrogen, unsubstituted C₁-C₁₀ alkyl, substituted C₁-C₁₀ alkyl, substituted C₂-C₁₀ alkenyl, unsubstituted C₂-C₁₀ alkenyl, substituted C₂-C10 alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl; and

R¹⁸ is hydrogen, acyl, sulfonyl or carbamoyl.

In another aspect of the present invention, compounds are provided of structure VIII wherein

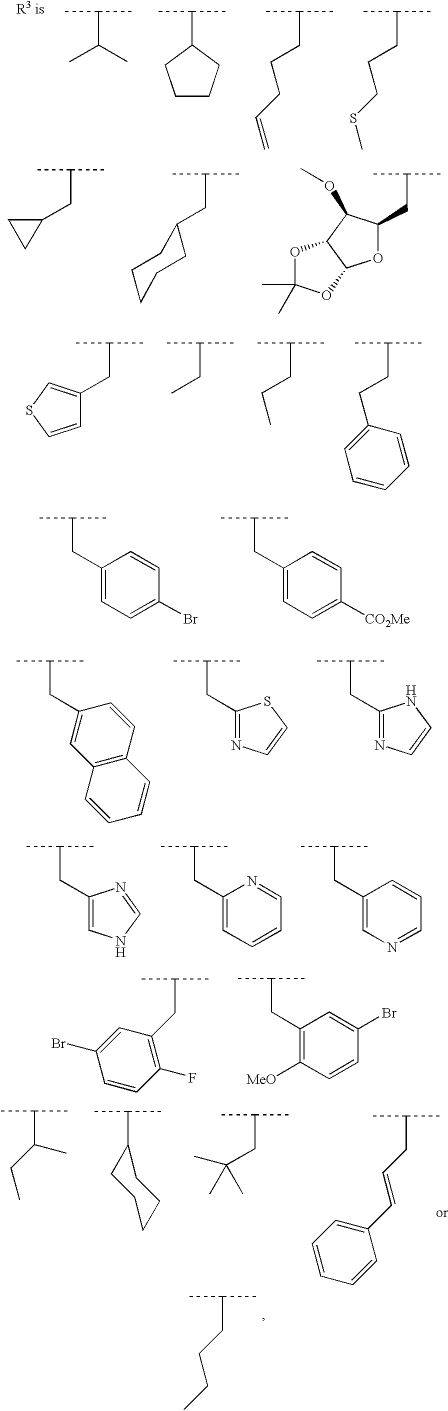

Starting Materials

The compounds of the present invention can be prepared in accordance with the methods of the present invention by a combination of recombinant DNA technology and organic chemistry.

Recombinant techniques are used to provide, in many instances, “unnatural” erythromycins or erythromycin derivatives that differ in one or more positions from the naturally occurring erythromycins A, B, C, or D. Although any suitable recombinant means may be used, a useful starting point is the complete 6-dEB synthase gene cluster that has been cloned in vectors and thus is amenable to genetic manipulations in E. coli and expression of the polyketide in Streptomyces. See U.S. Pat. Nos. 5,672,491; 5,830,750; 5,843,718; 5,712,146; and 5,962,290 which are all incorporated herein by reference. Once the aglycone is formed, it is next hydroxylated and/or glycoslyated and/or methylated at the appropriate positions by a converter strain that possesses the desired functionalities.

A particularly useful converter strain is an Saccharopolyspora erythraea eryA mutant that is unable to produce 6-dEB but can still carry out the desired conversions (Weber et al., J. Bacteriol. 164(1): 425-433 (1985). This mutant strain is able to take exogenously supplied 6-dEB and process it to erythromycin A by converting it into erythronolide B, 3-α-mycarosylerythronolide B, erythromycin D, erythromycin C, and finally to erythromycin A. An alternative route to erythromycin A is through erythromycin B where exogenously supplied 6-dEB is converted into erythronolide B, 3-α-mycarosylerythronolide B, erythromycin D, erythromycin B, and finally to erythromycin A. Other mutant strain, such as eryB, eryC, eryG, and/or eryK mutants, or mutant strains having mutations in multiple genes can be used to make compounds having any combinations of hydroxylations at C-6 and C-12, glycosylations at C-3 and C-5, and methylation at C-3″-OH. Any of these products may be used as starting materials for the practice of the present invention.

For erythromycins where the substituent at C-13 is methyl or ethyl, the 6-deoxyerythronolide B synthase (“DEBS”) from S. erythraea can be used in a recombinant expression system described in U.S. Pat. No. 5,672,491 to produce the aglycone in Streptomyces coelicolor. Optionally, the oleandolide or megalomicin polyketide synthase (“PKS”) genes may be used in this expression system. See U.S. Provisional Patent Application Serial No. 60/158,305 filed Oct. 8, 1999 and utility application Serial No. 09/679,279 filed Oct. 4, 2000 entitled Recombinant Megalomicin Biosynthetic Genes by inventors Robert McDaniel and Yana Volchegursky (Attorney Docket No. 30062-20047.20); and PCT Publication No. WO 00/026349 which are all incorporated herein by reference.

For erythromycins where the substituent at C-13 is something other than methyl or ethyl, one can employ a technique known as chemobiosynthesis in which activated thioesters called SNAC-diketides are converted to 13-substituted 6-dEB derivatives (13-R-13-desethyl-6-dEB compounds) by fermentation of S. coelicolor CH999/pJRJ2 or functionally similar strains that contain a PKS in which the ketosynthase domain of module 1 has been inactivated by mutation (the KS1° mutation). This methodology is described in PCT Publication Nos. WO 97/02358 and WO 99/03986 and U.S. Pat. No. 6,066,721 which are all incorporated herein by reference. Additional SNAC-diketide compounds and the corresponding aglycones are described in PCT Publication No. WO 00/44717 which is incorporated herein by reference. 6-dEB and 6-dEB derivatives such as 13-substituted 6-dEB are converted into the desired erythromycin starting material by an appropriate converter strain. For example, any one of the post PKS products may be used as starting materials such as 13-substituted counterparts (where the ethyl group which normally exists at C-13 is replaced with another substituent) to: erythronolide B, 3-α-mycarosylerythronolide B, erythromycin D, erythromycin B, erythromycin C, and erythromycin A. In particular, 13-substituted erythromycin A can be made by fermentation with an eryA mutant that is incapable of producing 6-dEB but can still carry out the desired conversions. 13-substituted erythromycin B can be made by fermentation with an eryA mutant that is incapable of producing 6-dEB and in which the ery K (12-hydroxylase) gene has been deleted or otherwise rendered inactive. Alternatively, erythromycin B derivatives can be made in a KS1°/eryK mutant strain of S. erythaea. The general method for using chemobiosynthesis for making modified 6-dEB is illustrated by Example 1 with specific reference to 13-propyl-6-dEB (13-propyl-13-desethyl-6-dEB). The general method for converting modified 6-dEB compounds to the desired hydroxylated and glycosylated form by using an eryA converter strain is illustrated by Example 2 with specific reference to converting 13-propyl 6-dEB to 13-propyl erythromycin A (13-propyl-13-desethyl-erythromycin A).

6-Desmethyl erythromycins, a starting material for making the furanyl erythromycins (compounds of formula II or IV) of the present invention, are made by replacing the acyl transferase (“AT”) domain of module 4 (encoding a 6-methyl group) of a 6-dEB or 8,8a -deoxyoleandolide synthase with a malonyl specific AT domain (encoding a 6-hydrogen) to provide the 6-desmethyl analog of the erythromycin aglycone. Illustrative examples of malonyl specific AT domains include AT2 and AT12 of rapamycin; AT3 and AT4 of epothilone; and AT10 of FK-520.

Alternatively, the AT4 domain of 6-dEB or 8,8a-deoxyoleandolide polyketide synthase is mutated to correspond to AT domains more characteristic of AT domains having malonyl specificity. More particularly, three mutations are made. In the first, nucleotides 6214-6227 of the open reading frame encoding AT4 (CGC GTC GAC GTG CTC) is modified to the sequence, GAC GAC CTC TAC GCC where bold indicates the altered nucleotide, to change the encoded amino acids from RVDVLQ to DDLYA. In the second, nucleotides 6316-6318 (CAG) is modified to the sequence CTC to change the encoded amino acid from Q to L. In the third, nucleotides 6613-6621 (TAC GCC TCC) is modified to the sequence CAC GCC TTC to change the encoded amino acids from YAS to HAF.

In either case, the resulting aglycone is bioconverted to 6-desmethyl erythromycin as described above although some modification for C-6 hydroxylation may be required.

Other starting materials include 6-hydroxy-erythromycin (where the methyl at C-6 has been replaced with a hydroxyl group), 6-oxo erythromycin (where the methyl at C-6 has been replaced with an oxo group), 6-methoxy erythromycin (where the methyl at C-6 has been replaced with a methoxy group) and 6-desmethyl, 7-hydroxy-erythromycin. In one embodiment, 6-OH, 6-OMe erythromcyins are made by replacing AT4 of 6-dEB or 8,8a -deoxyoleandolide synthase with an AT domain encoding hydroxymalonate or methoxymalonate. See PCT Publication WO 00/20601 which is incorporated herein by reference. The 6-OH and 6-OMe aglycone is bioconverted to 6-desmethyl-6-hydroxy erythromycin and 6-desmethyl-6-methoxy erythromycin respectively by fermentation with an appropriate eryA mutant that is incapable of producing 6-dEB and in which the eryF (C-6 hydroxylase) function has been deleted or otherwise inactivated. Fermentation of 6-OH or 6-OMe aglycone with an eryA mutant that possesses eryF (or equivalent) function leads to the 6-desmethyl-6-oxo erythromycin.

In one embodiment, 6-desmethyl, 7-hydroxy erythromycins are made by replacing AT4 of a 6-dEB or 8,8a-deoxyoleandolide polyketide synthase with a malonyl specific AT as described above as well as deleting or otherwise inactivating the dehydratase activity of module 3 (“DH3”). The resulting 6-desmethyl, 7-hydroxy aglycone is converted into the corresponding erythromycin derivative by fermentation with an appropriate eryA mutant that is incapable of producing 6-dEB as described above.

Synthetic Methods

The methods described herein are generally applicable to erythromycins and their derivatives unless explicitly limited. As such, references to specific embodiments are for the purposes of illustration only and are not intended to limit in any way the scope of the present invention.

In one aspect of the present invention, methods for forming the major types of intermediate compounds that are subsequently converted to the corresponding lactams are provided. These intermediate compounds include the 6,9-enol-ether, 6,9-epoxide, and furanyl erythromycins. The 6,9-enol ether erythromycins are also referred to as 8,9-anhydro erythromycin 6,9-enol ethers, enol ethers or dihydrofurans. The 6,9-epoxides are also referred to as epoxides or tetrahydrofurans.

Scheme 1A illustrates one embodiment for making the enol ether and epoxide compounds from erythromycin A derivatives (where R⁷ is as previously described).

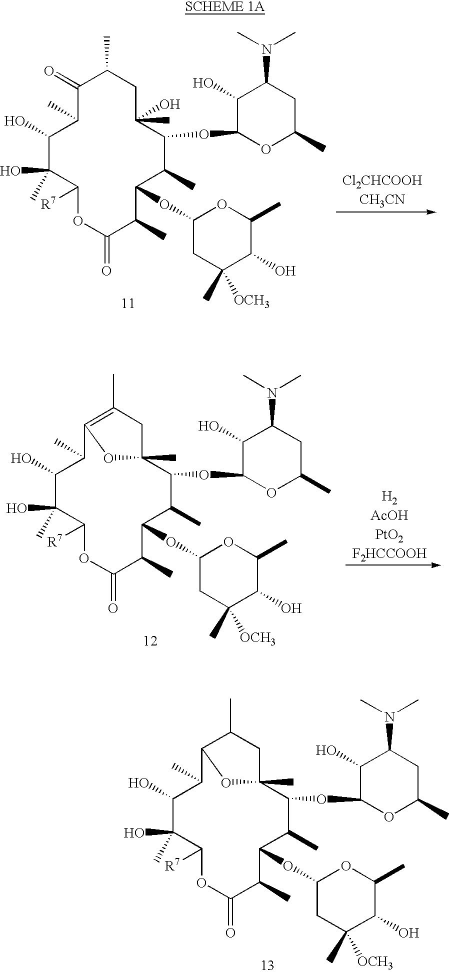

Enol ether compounds 12 are formed by treating with mild acid the desired erythromycin starting material such as 11. The corresponding epoxide 13 is formed by reducing the carbon-carbon double bond between C-8 and C-9 of the enol ether 12. Scheme 1B illustrates another embodiment for making epoxide 13.

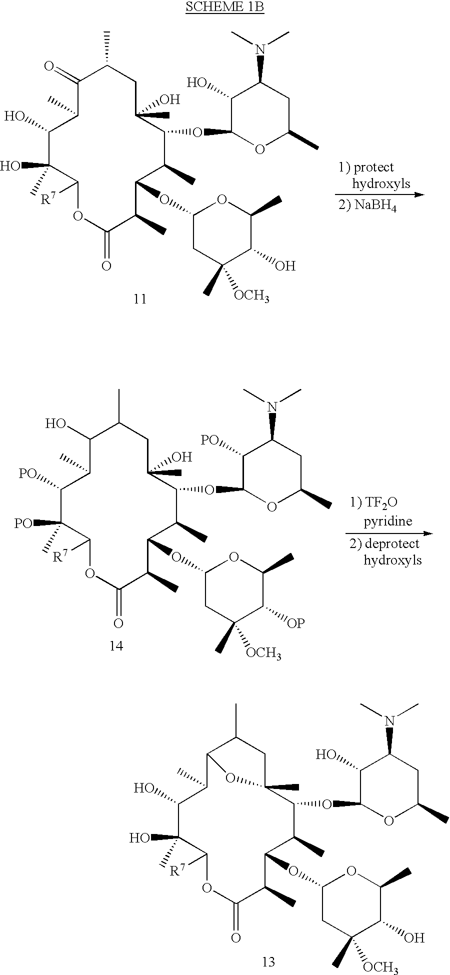

The free hydroxyls of erythromycin 11 are protected and the C-9 oxo is reduced with sodium borohydride to a 9-dihydro erythromycin intermediate 14 (where C-9 is —CHOH—). Illustrative examples of suitable protecting groups include acetyl for the C-2′, a carbonate ester such as Troc, Cbz or Boc for C-4″ hydroxyls and a cyclic carbonate for the C-11 and C-12 hydroxyls. The hydroxyl group at C-9 of compound 14 is subsequently activated and displaced to form epoxide 13. In one embodiment, the epoxide is formed by treatment with triflic anhydride and pyridine.

Furanyl erythromycins may be prepared using several different strategies. In one embodiment, furanyl erythromycins are prepared synthetically by demethylating the naturally occurring methyl group at C-6. For example, a suitably protected erythromycin is converted to the 6-O-xanthate via reaction with carbon disulfide and methyl iodide, and the xanthate is pyrolyzed to yield 6,6a-anhydroerythromycin. Ozonolysis yields the 6-oxo-erythromycin, which can be converted to the 6,9-epoxide by dehydration from treatment with mild acid or acetic anhydride. Alternatively, the 6-oxo-erythromycin may be prepared recombinantly as described previously. Scheme 2 illustrates another embodiment using 6-desmethyl erythromycins.

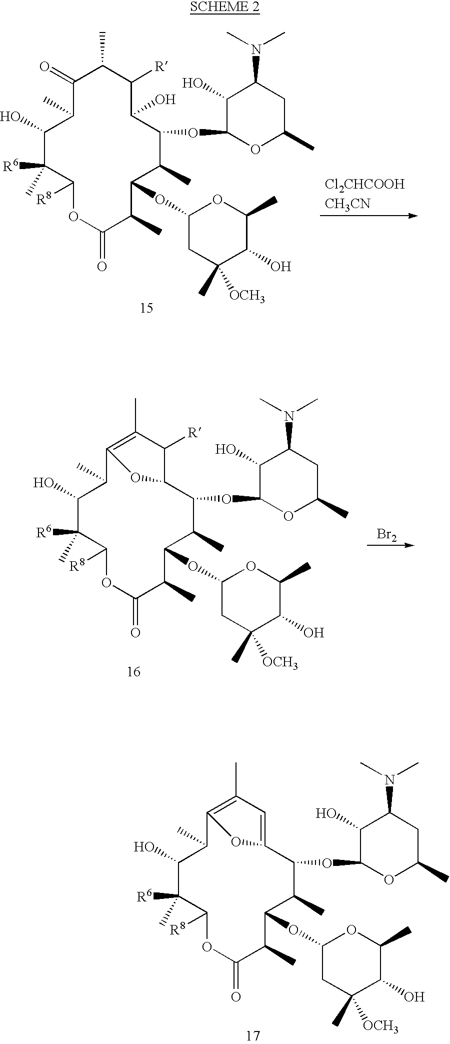

6-Desmethyl erythromycin 15 (where R′ and R⁶ are hydrogen or hydroxyl and R⁸ is as previously described) is treated with mild acid such as dichloroacetic acid to form enol ether 16. Compound 16 is then treated with a mild oxidizing agent such as bromine in base to yield furanyl erythromycin 17. In yet another embodiment, 6-desmethyl-7-hydroxy-8,9-anhydro erythromycin 6,9-hemiacetal is (specific embodiment of compound 16 where R′ is hydroxyl) is mesylated and subjected to base-catalyzed elimination to yield furanyl erythromycin 17.

In another aspect of the present invention, methods for converting the enol ether, epoxide and furanyl intermediate to the corresponding lactams are provided. In one embodiment, erythromycin lactams are made from 6,9-enol ethers (17 where x is a double bond) and 6,9-epoxides (17 where x is a single bond) as shown by Scheme 3A.

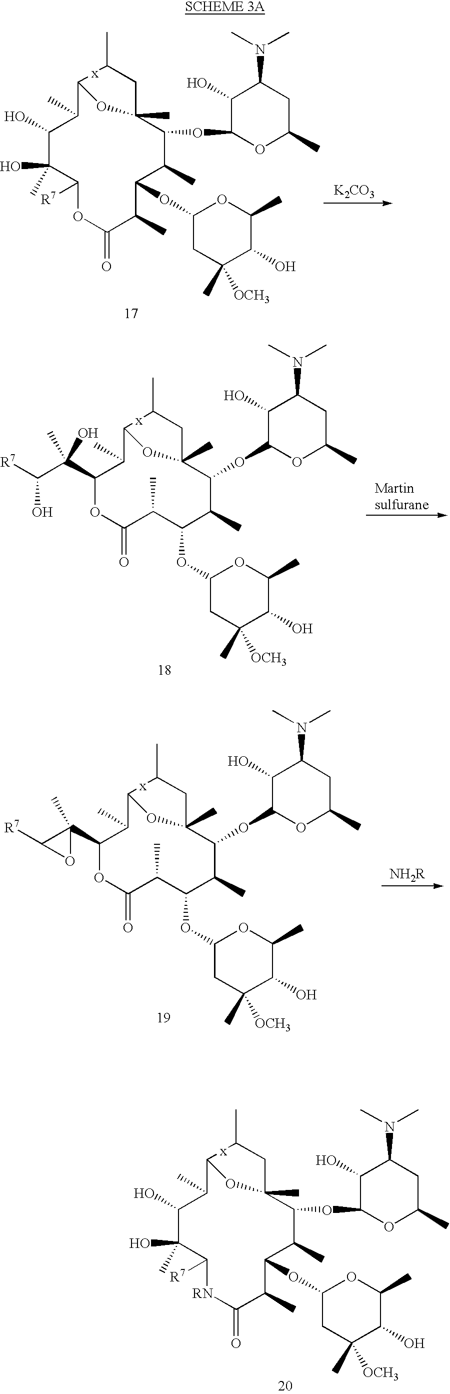

Compound 17 is treated with potassium carbonate in methanol to form the 12 membered derivative 18 which is converted into 12, 13 epoxide 19 by treatment with Martin sulfurane. Compound 19 is reacted with NH₂R to form erythromycin lactam 20 (where R is as described previously). 6,9 enol ether lactam compounds are 20 where x is a double bond and 6,9-ether lactams are 20 where x is a single bond.

As illustrated by Scheme 3B, furanyl erythromycin lactams are made similarly.

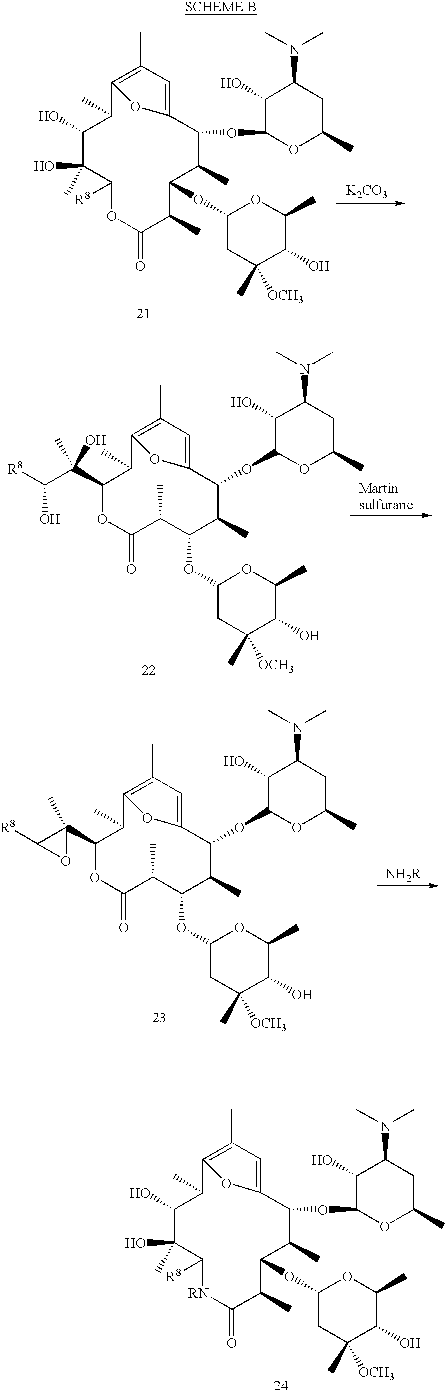

Furanyl erythromycin 21 (where R⁸ is as described previously) is treated with potassium carbonate to form the 12 membered derivative 22 which is converted into 12, 13 epoxide 23 by treatment with Martin sulfurane. Compound 23 is reacted with NH₂R to form furanyl erythromycin lactam 20 (where R is as described previously).

Derivatives of lactams 20 and 24 may be made by making the desired modifications either before or after lactam formation. In most cases, the timing of the modifications is based on synthetic convenience.

In another aspect of the present invention, methods for making 3′-N-desmethyl-3′N-alkyl lactam compounds are provided. One or both of the 3′-N-methyl groups are demethylated and the demethylated 3′-nitrogen is subsequently reacted with a substituted or unsubstituted alkyl or aryl group. The 3′-N demethylation and subsequent alkylation (or arylation) may be performed using erythromycins, enol ethers, epoxide and furanyl erythromycins as well as their lactam counterparts. Scheme 4 illustrates one embodiment where the demethylation and alkylation reactions are illustrated with respect to 6,9-enol ether 12.

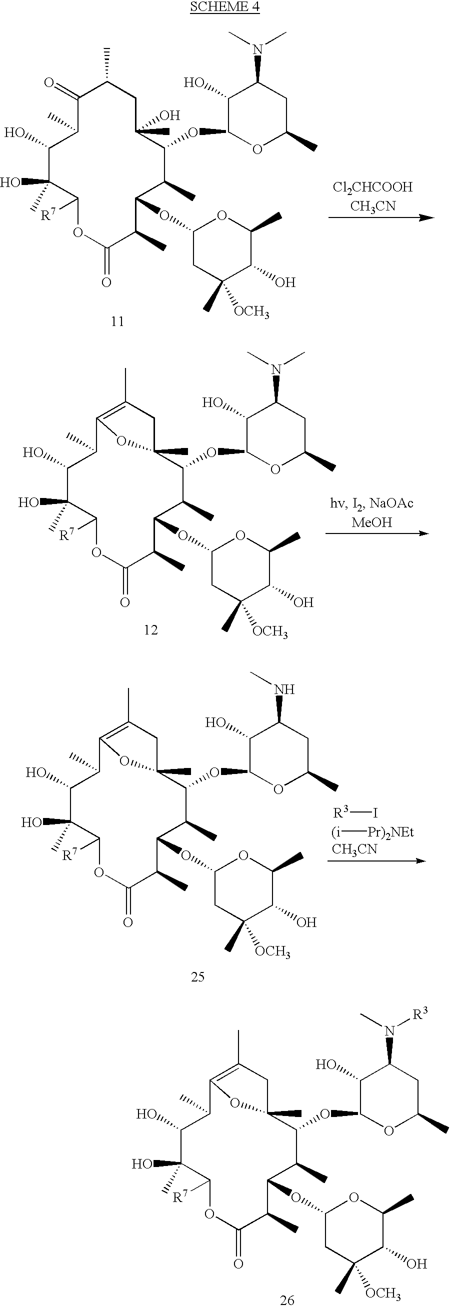

Enol ether 12, formed from erythromycin 11 as described previously by Scheme 1A, is demethylated at the 3′-N by treatment with light, iodine and sodium acetate. Additional reagents and longer reaction times will remove both methyl groups if desired. The demethylated enol ether 25 is then alkylated or arylated with the appropriate alkyl halide or aryl halide to yield compound 26. Enol ether 26 may be optionally reduced to form its 6,9 epoxide counterpart using the procedures described by Scheme 1A. Compound 26 or its epoxide counterparts are used as starting materials for the protocols described in Scheme 3 to make the corresponding 3 ′-N-desmethyl-3′-N-alkyl lactams.

In another aspect of the present invention, methods for making 4″-desoxy lactams are provided. In one embodiment, 4″-desoxy erythromycin is made as described by Scheme 5.

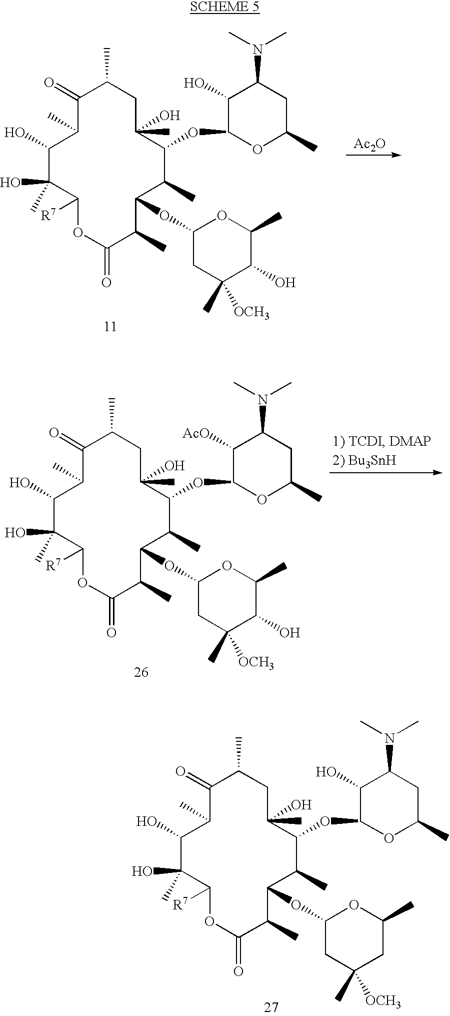

Erythromycin 11 is acetylated at the 2′ hydroxyl to yield compound 26. The 2′-O-acetyl erythromycin 26 is then treated with thiocarbonyldiimidazole and 4-dimethylaminopyridine in dichloromethane. The resulting product is isolated and treated with tributyltin hydride to yield compound 27. The corresponding lactam can be made by using compound 27 as starting materials in the protocols described by Schemes 1 and 3.

In another aspect of the present invention, an alternate route for making erythromycin lactam is provided.

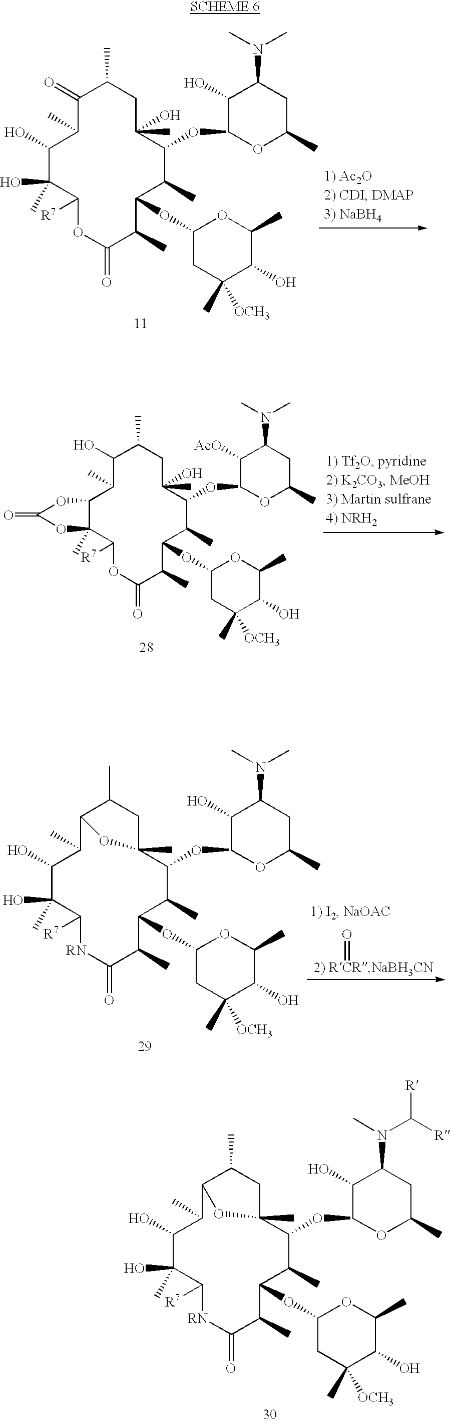

As shown in Scheme 6, erythromycin 11 is converted into 2′-O-acetyl-9-dihydro -erythromycin A 11, 12, cyclic carbamate 28 which is transformed into the corresponding 6,9-epoxide The 6,9-epoxide is converted into lactam 29 as previously described in Scheme 3. Compound 29 may then be subsequently modified using standard procedures that are known in the art. See e.g. Advanced Organic Chemistry 3rd Ed. by Jerry March (1985) which is incorporated herein by reference. For example, the 3′-N-methyl group may be demethylated and subsequently modified by reductive amination to yield compound 30. Alternatively, a keto group may be formed at C-11 by protecting the 2′, 4″ and 12 hydroxyls and oxidizing the C-11 hydroxyl to a ketone.

In another aspect of the present invention, methods for making 3′-desmethyl erythromycin oximinoester are provided. One embodiment is shown in Scheme 7.

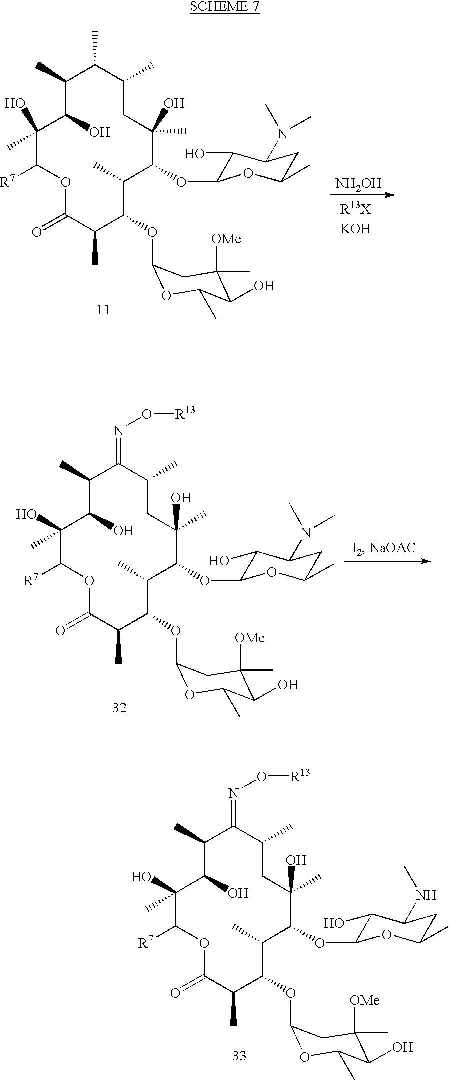

As shown in Scheme 7, erythromycin 11 is converted into the 9-oximinoether 32 using an O-alkyl or O-aryl hydroxylamine. Alternatively oximinoethers 32 can be prepared by alkylation of erythromycin 9-oxime. One of the 3′ dimethyl groups of the desosamine sugar is then demethylated with iodine to yield compound 33.

In another aspect of the present invention, methods for converting the 3′-desmethyl erythromycin oximinoester into 3′-desmethyl-R erythromycin oximinoesters are provided. Two embodiment are illustrated in Scheme 8.

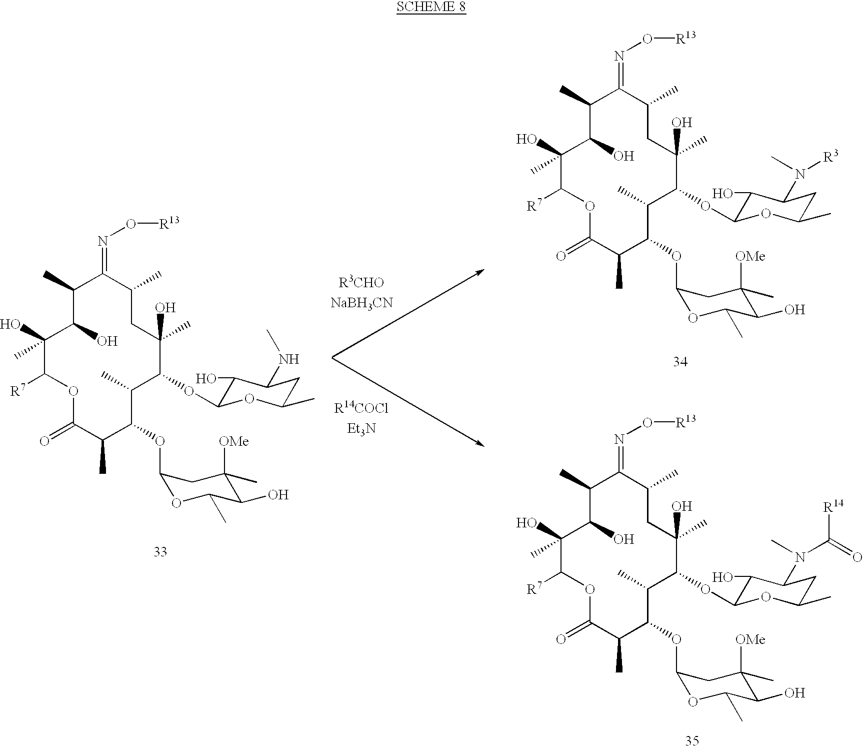

As shown in Scheme 8, in the first route, reductive alkylation of 33 using an aldehyde or ketone in the presence of NaBH₃CN introduces R³ as a substituted or unsubstituted alkyl, alkenyl, or aryl group. In the second route, the 3′-amino group of 33 is converted to amide, carbamate or urea through reaction with the appropriate acyl halide R14COCl.

In another aspect of the present invention, methods for modifying the 4″ hydroxyl group of the cladinose are provided. One embodiment is illustrated in Scheme 9.

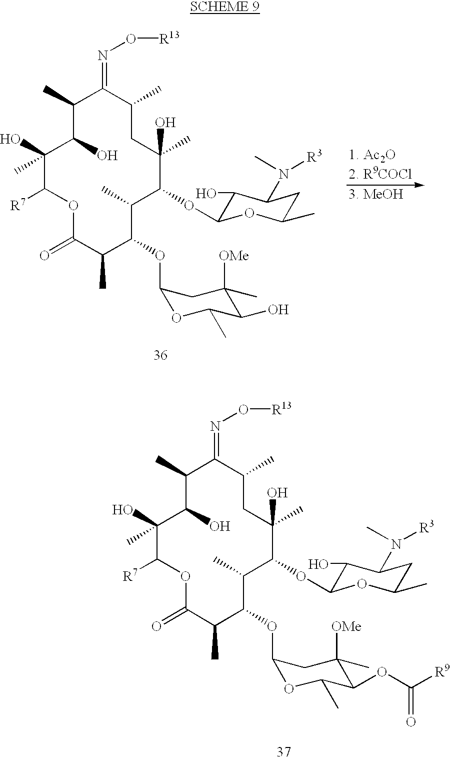

As shown by Scheme 9, the 2′ hydroxyl of the desosamine is protected with a protecting group such as acetyl and the 4″ hydroxyl of the cladinose is functionalized using for example, an acid chloride in the presence of a base such as DMAP. For example, compound 36 is used in this illustration to make the corresponding 4″modified compound. Deprotection with methanol yields the desired product which in this case is compound 37.

In another aspect of the present invention, methods for removing the cladinose are provided. One embodiment is illustrated by Scheme 10.

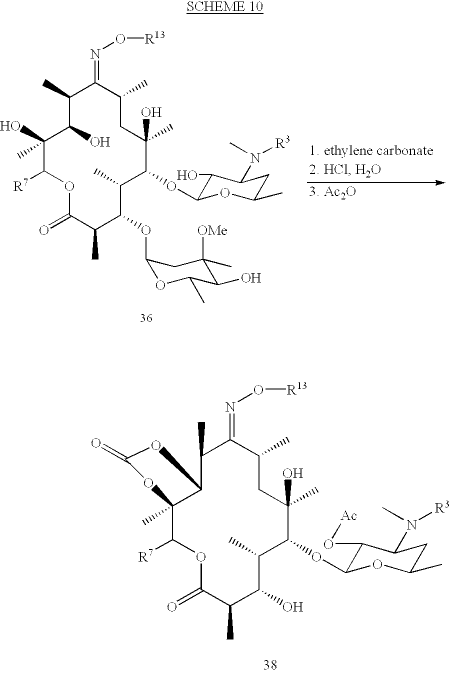

As shown in Scheme 10, the 10, 11-diol of a compound, such as compound 36 in this example, is protected through carbonate exchange with ethylene carbonate and the cladinose moiety is removed by mild acid hydrolysis (0.5 N HCl). If further chemistry is desired at the resulting C-3 hydroxyl, the 2′ hydroxyl is transiently protected with a protecting group such as an acetyl group to yield compound 38.

In another aspect of the present invention, methods for modifying the C-3 hydroxyl are provided. Two embodiments of this method are illustrated in Scheme 11.

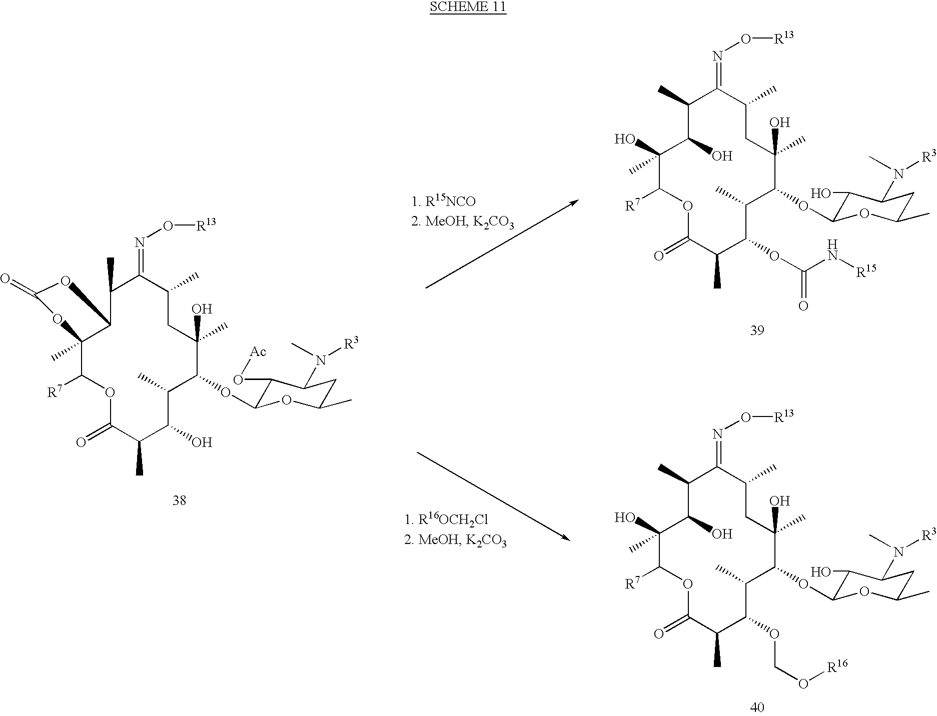

As shown in Scheme 11, a suitably protected compound such as 38 can be functionalized through carbamoylation to yield compound 39 or through formation of a mixed acetal to yield compound 40.

Methods of Use

In general, methods of using the compounds of the present invention comprise administering to a subject in need thereof a therapeutically effective amount of a compound of the present invention. Illustrative examples of disorders that may be treated with the inventive compounds include but are not limited to gastroparesis, gastroesophageal reflux disease, anorexia, gall bladder stasis, postoperative paralytic ileus, scleroderma, intestinal pseudoobstruction, gastritis, emesis, and chronic constipation (colonic inertia).

The therapeutically effective amount can be expressed as a total daily dose of the compound or compounds of this invention and may be administered to a subject in a single or in divided doses. The total daily dose can be in amounts, for example, of from about 0.01 to about 25 mg/kg body weight, or more usually, from about 0.1 to about 15 mg/kg body weight. Single dose compositions may contain such amounts or submultiples thereof as to make up the daily dose. In general, treatment regimens according to the present invention comprise administration to a subject in need of such treatment of from about 10 mg to about 1000 mg of the compound(s) of the present invention per day in single or multiple doses.

Typically, the inventive compound will be part of a pharmaceutical composition or preparation which may be in any suitable form such as solid, semisolid, or liquid form. In general, the pharmaceutical preparation will contain one or more of the compounds of the invention as an active ingredient and a pharmaceutically acceptable carrier. Typically the active ingredient is in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. Oral dosage forms may be prepared essentially as described by Hondo et al., 1987, Transplantation Proceedings XIX, Supp. 6: 17-22, incorporated herein by reference.

The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used. For example, the compounds of the invention may be utilized with hydroxypropyl methylcellulose essentially as described in U.S. Pat. No. 4,916,138, incorporated herein by reference, or with a surfactant essentially as described in EPO patent publication No. 428,169, incorporated herein by reference.

In summary, the present invention provides novel macrolide compounds, methods for making and methods of using the same which are further illustrated by the following examples.

### EXAMPLE 1

### Method of Making 13-propyl-6-deoxyerythronolide B (13-propyl-6-dEB

A 1 mL vial of the CH999/pJRJ2 (Streptomyces coelicolor that contains a PKS in which the ketosynthase domain of module 1 has been inactivated by mutation) working cell bank is thawed and the contents of the vial are added to 50 mL of Medium 1 in a 250 mL baffled flask.

Medium 1 comprises 45 g/L cornstarch; 10 g/L corn steep liquor; 10 g/L dried, inactivated brewers yeast; and 1 g/L CaCO₃. This solution is sterilized by autoclaving for 90 minutes at 121° C. After sterizilization, 1 mL/L of sterile filtered 50 mg/ml thiostrepton in 100% DMSO and 1 mL/L autoclaved 100% antifoam B silicon emulsion (J. T. Baker) are added prior to use.

The flask is placed in an incubator/shaker maintained at 30±1° C. and 175±25 RPM for 48±10 hours. The 50 mL culture is then added to a 2.8 L baffled flask containing 500 mL of Medium 1. This flask is incubated in an incubator/shaker at 30±1° C. and 175±25 RPM for 48±10 hours. The 500 mL culture is than used to inoculate a 10 L fermenter containing 5 L of Medium 1. The fermenter is controlled at 30° C., pH 6.5 by addition of 2.5 N H₂SO₄ and 2.5 N NaOH, agitation rate 600 RPM, and air flow rate 1-2 LPM. Foam is controlled by the addition of a 50% solution of Antifoam B as needed. The fermenter culture is allowed to grow under these conditions for 24±5 hours.

A 150 L fermenter is prepared by sterilizing 100 L of Medium 1 at 121 ° C. for 45 minutes. After the growth period, the contents from the 10 L fermenter are aseptically added to a 150 L fermenter. The fermenter is controlled at 30° C., pH 6.5 by addition of 2.5 N H₂SO₄ and 2.5 N NaOH, dissolved oxygen ≧80% air saturation by agitation rate (500-700 RPM), air flow rate (10-50 LPM), and/or back pressure control (0.1-0.4 bar). Foam is controlled by the addition of a 50% solution of Antifoam B as needed.

At 35±5 hours, after dissolved oxygen has reached a minimum and CO₂ content in fermenter offgas has reached a maximum, (2S, 3R)-2-methyl-3-hydroxypentanoyl-N-acetylcysteamine (propyl diketide) is added to a final concentration of 2 g/L. Propyl diketide is prepared by solubolizing in dimethyl sulfoxide at a ratio of 2:3 (diketide to DMSO) and then filter sterilized (0.2 μm, nylon filter). Production of 13-propyl-6-deoxyerythonolide B (13-propyl-6dEB) ceases on day 8 and the fermenter is harvested. The fermentation broth is centrifuged at 20,500 g in an Alpha Laval AS-26 centrifuge. The product is predominantly in the centrate; the centrifuged cell mass is discarded.

After centrifugation, solid phase extraction is performed using HP20 resin (Mitsubishi). Column size is selected based on centrate volume and titer, so that the loading capacity of 15 g 13-propyl-6-dEB per liter HP20 resin is not exceeded. The centrifuged broth is passed through the resin bed at a linear flow rate of 300±20 cm/h. The pressure on the column should not exceed 15 psi. The resin is then washed with 2 column volumes (CV) of water and then 2 CV of 30% methanol, each at a rate of 300±20 cm/h. 13-propyl-6dEB is eluted using 7-10 CV 100% methanol at a rate of 300±20 cm/h. During elution, fractions of ½ CV are collected. The fractions are then analyzed, and those containing product are combined to yield a product pool containing >95% of the original 13-propyl-6-dEB in the centrifuged broth. The product pool is reduced to solids using rotary evaporation. Product purity at this stage is 5-35%. Methanol-insoluble material is removed from the product pool by suspending the solids in 3 L 100% methanol per 100 L original broth volume, mixing for 20 minutes, and filtering.

The final purification step is chromatography using HP20SS resin (Mitsubishi). Column size is selected based on amount of product, so that the loading capacity of 15 g 13-propyl-6-dEB per liter HP20SS resin is not exceeded. The filtered methanol solution is diluted by adding an equal volume of water. The 50% methanol solution is passed through the resin bed at a linear flow rate of 300±20 cm/h. The column is then washed with 2 CV of 50% methanol at a rate of 300±20 cm/h. Product is eluted using 12 CV 70% methanol at a rate of 300±20 cm/h. During elution, fractions of ½ CV are collected. The fractions are then analyzed, and those containing >50 mg/L 13-propyl-6-dEB and having >20% chromatographic purity are combined. The product pool is reduced to solids using rotary evaporation. Product purity at this stage is >65% and is suitable for bioconversion to the appropriate erythromycin.

### EXAMPLE 2

### Method of Making 13-propyl Erythromycin A

A 1 mL vial from working cell bank K39-14V (an eryA mutant of S. erythraea that is incapable of producing 6-dEB) is thawed and the contents of the vial are added to 50 mL of Medium 2 in a 250 mL baffled flask.

Medium 2 comprises 16 g/L cornstarch; 10 g/L corn dextrin; 15 g/L soy meal flour; 4 g/L CaCO₃; 5 g/L corn steep liquor; 6 g/L soy bean oil; 2.5 g/L NaCl; and 1 g/L (NH₄)₂SO₄. This solution is sterilized by autoclaving for 60 minutes at 121° C. and 1 mL/L autoclaved 100% antifoam B silicon emulsion (J. T. Baker) is added prior to use.

The flask is placed in an incubator/shaker maintained at 34±1° C. and 175±25 RPM for 48±10 hours. The 50 mL culture is then added to a 2.8 L baffled flask containing 500 mL of Medium 2. The flask is incubated in an incubator/shaker at 34±1° C. and 175±25 RPM for 48±10 hours. The 500 mL culture is than used to inoculate a 10 L fermenter containing 5 L of Medium 2. The fermenter is controlled at 34° C., pH 7.0 by addition of 2.5 N H₂SO₄ and 2.5 N NaOH, agitation rate 600 RPM, and air flow rate 1-2 LPM. Foam is controlled by the addition of a 50% solution of Antifoam B as needed. The fermenter culture is allowed to grow under these conditions for 24±5 hours.

A 150 L fermenter is prepared by sterilizing 100 L of Medium 3 at 121° C. for 45 minutes. Medium 3 comprises 17.5 g/L cornstarch; 16 g/L corn dextrin; 16.5 g/L soy meal flour; 4 g/L CaCO₃; 6 g/L corn steep liquor; 3 g/L soy bean oil; 3.5 g/L NaCl; and 1 g/L (NH₄)₂SO₄. After the growth period, the contents from the 10 L fermenter are aseptically transferred to the 150 L fermenter. The fermenter is controlled at 34° C., pH 7.0 by addition of 2.5 N H₂SO4 and 2.5 N NaOH, dissolved oxygen ≧80% air saturation by agitation rate (500-700 RPM), air flow rate (15-50 LPM), and/or back pressure control (0.1-0.4 bar). Foam is controlled by the addition of a 50% solution of Antifoam B.

At 24±5 hours a 58-60 mL/hour 15% dextrin (w/v) feed is initiated. The dextrin solution is continuously mixed during the feed period. At 24±5 hours 25 grams of 13-propyl-6dEB are added to the fermenter. The 13-propyl-6dEB is prepared by solubolizing 25 grams of 13-propyl-6dEB in 400-600 mL of 100% ethanol and filtering (0.2 μm, nylon filter). Conversion of 13-propyl-6dEB to 13-propyl-erythromycin A ceases after 60±10 hours and the fermenter is harvested. The fermentation broth is centrifuged at 20,500 g in an Alpha Laval AS-26 centrifuge. The product is predominantly in the centrate; the centrifuged cell mass is discarded.

After centrifugation, solid phase extraction is performed using HP20 resin (Mitsubishi). Column size is selected based on centrate volume and titer, so that the loading capacity of 15 g 13-propyl-erythromycin A per liter HP20 resin is not exceeded. The centrifuged broth is adjusted to pH 9, then passed through the resin bed at a linear flow rate of 275±25 cm/h. The pressure on the column should not exceed 15 psi. The resin is then washed with 1 column volume (CV) of water at a rate of 275±25 cm/h. 13-propyl-6dEB is eluted using 5 CV 100% methanol at a rate of 275±25 cm/h. During elution, fractions of 1 CV are collected. The fractions are then analyzed, and those containing product are combined to yield a product pool. The product pool is reduced to solids using rotary evaporation.

Methanol-insoluble material is removed from the product pool by suspending the solids in 1L 100% methanol per 100 L original broth volume, adjusting to pH 9, and filtering. The product pool (filtrate) is reduced to solids using rotary evaporation.

13-propyl-erythromycin A is extracted from the product pool (solids) by adding 2 L 4:1 hexane:acetone per 100 L original broth volume, mixing for 20 minutes, and filtering. The remaining solids are extracted the same way two more times and filtrates are combined. The product pool is reduced to solids using rotary evaporation.

The final purification step is chromatography using HP20SS resin (Mitsubishi). Column size is selected based on amount of product, so that the loading capacity of 15 g 13-propyl erythromycin A per liter HP20SS resin is not exceeded. The solids from the previous steps are dissolved in 1 L methanol per 100 L original broth volume, and an equal volume of water is added. The 50% methanol solution is passed through the resin bed at a linear flow rate of 275±25 cm/h. The column is then washed with 1 CV of 50% methanol, then 3 CV 60% methanol, each at a rate of 275±25 cm/h. Product is eluted using 3 CV 70% methanol, then 10 CV 75% methanol, each at a rate of 275±25 cm/h. During elution, fractions of ½ CV are collected. The fractions are then analyzed, and those containing 13-propyl-erythromycin A are combined. The product pool is reduced to solids using rotary evaporation.

### EXAMPLE 3

### 8,9-anhydro-6,9-hemiacetal (enol ether) formation

A solution of erythromycin (100 mg) in anhydrous acetonitrile (2 mL) is treated with dichloroacetic acid (0.015 mL) under inert atmosphere until thin-layer chromatography reveals disappearance of starting material (2 days). The reaction mixture is concentrated, redissolved in 50 mL of dichloromethane, and washed with saturated NaHCO₃. The organic phase is dried over Na₂SO₄, filtered, and concentrated to give the crude product. Silica gel chromatography (acetone +2% Et₃N, hexanes) gives the pure product. Other compounds of the invention are formed by substituting the corresponding erythromycin derivative for the erythromycin in the above procedure.

An exemplary NMR data for one of the compounds of the present invention, 8,9-anhydroerythromycin A 6,9-hemiacetal is as follows. ¹³C-NMR (CDCl₃):δ178.2, 151.7, 102.9, 101.4, 94.6, 85.5, 80.1, 78.2, 78.1, 76.3, 75.3, 73.0, 70.8, 70.1, 68.8, 65.8, 65.6, 49.5, 44.7, 43.2, 42.6, 40.3, 34.6, 30.5, 28.7, 26.2, 21.5, 21.3, 21.0, 18.2, 16.1, 15.0, 13.4, 11.9, 11.4, 10.8, 8.6.

### EXAMPLE 4

### Hydrogenation of 8,9-anhydroerythromycin 6,9-hemiacetals to (8S,9R)-9-deoxo-6,9-epoxyerythromycins

A solution of the 8,9-anhydroerythromycin 6,9-hemiacetal (0.55 mmol; Example 3) in 24 mL of glacial acetic acid is treated with difluoroacetic acid (0.1 mL) and platinum oxide (0.4 g). The mixture is shaken under 4 atm of hydrogen at ambient temperature for 3 hours, or until consumption of starting material as indicated by thin-layer chromatography. Ammonium acetate (0.3 g) is added, the mixture is stirred for 15 minutes, then filtered and concentrated. The residue is dissolved in dichloromethane, washed with sat. NaHCO₃, dried over Na₂SO₄, filtered, and evaporated. Silica gel chromatography (acetone +2% Et₃N, hexanes) gives the pure product.

### EXAMPLE 5

### Ring contraction of 14-membered to 12-membered Macrolides

A solution of the 8,9-anhydroerythromycin 6,9-hemiacetal derivative (1 mmol; Example 3) and potassium carbonate (200 mg) in methanol (50 mL) is heated at reflux until thin-layer chromatographic analysis reveals the reaction has reached equilibrium. The mixture is evaporated to dryness, then dissolved in CH₂Cl₂ and chromatographed on silica gel. Both 14-membered enol ethers and 9-deoxo-6,9-epoxides are converted into their 12-membered macrolide counterparts using this procedure. Those derivatives containing 2′-O-acetates, 4″-O-formates, 4″-O-(2,2,2-trichloroethoxycarbonyl), or 11,12-cyclic carbonates are deprotected during this process.

### EXAMPLE 6

### 3′-N-desmethyl Erythromycin Derivatives

Sodium acetate trihydrate (300 mg) and iodine (116 mg) are added sequentially to a solution of erythromycin (300 mg) in 3 mL of methanol. The reaction mixture is exposed to a 120 W flood lamp and stirred until complete reaction is determined by thin-layer chromatographic analysis. Excess reagents are quenched by addition of saturated sodium thiosulfate solution, and the volatiles are removed under reduced pressure and the mixture is diluted with dichloromethane. The organic phase is washed with saturated NaHCO₃, dried over Na₂SO₄, filtered, and concentrated to give the crude product. Silica gel chromatography (acetone +2% Et₃N, hexanes) gives the pure product.

The 3′-N-desmethyl-8,9-anhydroerythromycin 6,9-hemiacetals are prepared by substituting the 8,9-anhydroerythromycin 6,9-hemiacetals for the erythromycin in the above procedure.

### EXAMPLE 7

### 3′-N-desmethyl-3′-N-alkyl-erythromycin Derivatives

A solution of the 3′-N-desmethyl-erythromycin derivative (0.5 mmol; Example 6) in acetonitrile (6 mL) is treated with diisopropylethylamine (0.23 mL) and the desired alkylating agent (0.6 mmol) and stirred at 40-80° C. under inert atmosphere until consumption of the erythromycin starting material as determined by thin-layer chromatographic analysis. The reaction mixture is concentrated under vacuum and redissolved in dichloromethane, washed with saturated NaHCO₃, dried over Na₂SO₄, filtered, and concentrated to give the crude product. Silica gel chromatography (acetone+2% Et₃N, hexanes) gives the pure product.

Alkylating agents useful in this procedure include ethyl iodide, benzyl bromide, 2-iodopropane, 4-bromo-1-butene, allyl bromide, propargyl bromide, or sec-butyl iodide, or the corresponding trifluoromethanesulfonates, which give rise to the 3′-N-ethyl, isopropyl, butenyl, allyl, propargyl, or sec-butyl derivatives, respectively.

3′-N-desmethyl-3′-N-alkyl-8,9-anhydroerythromycin 6,9-hemiacetal is prepared by substituting 3′-N-desmethyl-8,9-anhydroerythromycin 6,9-hemiacetal (Example 7) for the 3′-N-desmethyl-erythromycin in the above procedure.

### EXAMPLE 8

### 2′-O-acetyl-erythromycin

A 0° C. solution of erythromycin (13.4 mmol) in ethyl acetate (50 mL) is treated with acetic anhydride (1.4 mL) for 30 minutes, then kept for 4 hours at ambient temperature. The mixture is quenched with sat. NaHCO₃, and extracted with ethyl acetate. The extracts are combined, dried over MgSO4, filtered, and concentrated to dryness under reduced pressure to yield the crude product. The product is either crystallized or purified by silica gel chromatography. NMR data follows for one of the compounds of the present invention, 2′-O-acetyl-13-propyl erythromycin A, that was crystallized from acetonitrile. ¹³C-NMR (CDCl₃): δ222.3, 175.4, 170.0, 100.9, 96.1, 83.4, 79.7, 75.1, 75.0, 74.5, 72.7, 71.7, 68.9, 68.4, 65.7, 63.6, 49.4, 45.2, 44.8, 40.7, 39.2, 38.1, 37.8, 35.0, 31.6, 30.3, 30.2, 27.0, 22.6, 21.5, 21.5, 21.2, 19.5, 18.6, 18.1, 16.3, 15.8, 14.1, 14.0, 12.0, 9.0.

### EXAMPLE 9

### 2′-O-acetyl-4″-deoxy-erythromycin

Step 1. A mixture of 2′-O-acetyl-erythromycin (3.5 mmol; Example 8), thiocarbonyldiimidazole (1 g), and 4-dimethylaminopyridine (0.67 g) in 100 mL of CH₂Cl₂ is stirred overnight at ambient temperature. The mixture is treated with 150 mL of sat. NaHCO₃, and the organic phase is then washed with water, dried over MgSO₄, filtered, and evaporated. The product 4″-O-thiocarbonylimidazolide is crystallized.

Step 2. The product from Step 1 is dissolved in 60 mL of toluene and heated to 98° C. Tributyltin hydride (1.9 mL) is added followed by 2,2′-azobisisobutyronitrile (60 mg) and heating is continued for 35 minutes. The mixture is concentrated under reduced pressure. The oily residue is dissolved in 340 mL of acetonitrile, washed with 5 portions of hexanes, and concentrated to yield the crude product. Purification by silica gel chromatography yields the pure product. NMR data follows for one of the compounds of the present invention, 2′-O-acetyl-4″-deoxyerythromycin A: ¹³C-NMR: δ222.0, 175.6, 170.0, 100.4, 96.8, 83.2, 79.0, 74.8, 74.6, 71.7, 70.5, 68.9, 67.9, 63.2, 61.4, 49.2, 45.3, 45.1, 44.7, 40.7, 38.9, 37.9, 34.1, 30.7, 26.8, 25.5, 25.2, 22.2, 21.9, 21.5, 21.2, 18.2, 16.3, 15.9, 12.0, 10.6, 9.1.

### EXAMPLE 10

### 2′-O-acetyl-4″-O-(2,2,2,-trichloroethoxycarbonyl)-erythromycin

A solution of 2′-O-acetyl-erythromycin (100 mmol; Example 8) and 4-dimethylaminopyridine (49.0 g) in CH₂Cl₂ (500 mL) is cooled to −78° C. and stirred under inert atmosphere. Trichloroethyl chloroformate (50 mL) is added dropwise, and the mixture is stirred for 48 hours. After warming to ambient temperature, the mixture is washed with cold phosphate buffer (1:1 v/v mix of 5% KH₂PO₄ and 1% K₂HPO₄) followed by brine, dried over MgSO₄, filtered, and concentrated. The product is purified by crystallization or silica gel chromatography.

### EXAMPLE 11

### erythromycin A 11,12-cyclic Carbonate

A mixture of 2′-O-acetyl-4″-deoxy-erythromycin A (1.6 mmol; Example 9), 1,1-carbonyldiimidazole (1.64 g), and 4-dimethylaminopyridine (0.41 g) in 13 mL of CH₂Cl₂ is warmed gently to dissolve the solids, then allowed to stir overnight at ambient temperature. Saturated NaHCO₃ (20 mL) is added and stirred for 15 minutes, then the mixture is extracted with CH₂Cl₂. The extract is washed with water, dried over MgSO₄, filtered, and evaporated to yield 2′-O-acetyl-4″deoxy-erythromycin A 11,12-cyclic carbonate.

### EXAMPLE 12

### (9S)-9-dihydro-erythromycins

A solution of 2′-O-acetyl-4″-deoxy-erythromycin A 11,12-cyclic carbonate (0.5 mmol; Example 11) in 10 mL of ethanol is treated with sodium borohydride (200 mg), and the reaction is monitored by thin-layer chromatography. When the reaction is ca. 80% complete, 0.5 M phosphate buffer (50 mL) is added and the mixture is extracted with CH₂Cl₂. The extract is washed with phosphate buffer, dried over MgSO₄, filtered, and evaporated. The product, (9S)-2′-O-acetyl-4″-deoxy-9-dihydro-erythromycin A 11,12-cyclic carbonate, is purified by silica gel chromatography. NMR data follows for one of the compounds of the invention, (9S)-2′-O-acetyl-9-dihydroerythromycin A 11,12-cyclic carbonate: ¹³C-NMR: δ175.8, 169.9, 153.8, 100.1, 96.7, 85.3, 82.3, 81.1, 80.0, 77.7, 76.5, 74.6, 71.7, 70.6, 68.6, 62.9, 61.8, 49.1, 45.3, 44.7, 42.3, 40.7, 34.5, 34.2, 33.6, 30.9, 25.5, 25.1, 22.9, 21.5, 21.4, 21.0, 20.1, 14.5, 14.4, 14.3, 10.7, 9.2.

### EXAMPLE 13

### 9-deoxo-6,9-epoxy-erythromycins

A solution of (9S)-2′-O-acetyl-4″-deoxy-9-dihydro-erythromycin A 11,12-cyclic carbonate (1 mmol; Example 12) in 25 mL of CH₂Cl₂ at 0° C. is treated with pyridine (0.26 mL; Example 21) and trifluoromethanesulfonic anhydride (0.35 mL). After 30 minutes, sat. NaHCO₃ is added and the mixture is extracted with CH₂Cl₂. The extract is washed with water, dried over MgSO₄, filtered, and evaporated. The product, (8R,9R)-2′-O-acetyl-4″-deoxy-9-deoxo-6,9-epoxy-13-desethyl-13-R-erythromycin A, is isolated by silica gel chromatography.

### EXAMPLE 14

### Removal of 2′-O-acetate and 11,12-cyclic Carbonate Protection

A solution of the 2′-O-acetyl-erythromycin 11, 12-cyclic carbonate (1 mmol; Example 11) in 25 mL of methanol is treated with potassium carbonate (3 mmol). Upon completion of the reaction, the mixture is evaporated, and the residue is dissolved in CH₂Cl₂. The extract is washed with water, dried over MgSO₄, filtered, and evaporated. The product is isolated by silica gel chromatography.

### EXAMPLE 15

### Removal of 4″-O-(2,2,2-trichloroethoxycarbonyl) protection

Samarium iodide is prepared by stirring a solution of samarium (3.43 mmol) and iodine (3.09 mmol) in 40 mL of tetrahydrofuran at reflux for 2.5 hours. Upon cooling to ambient temperature, 10 mg of NiI₂ is added and the mix is cooled to −78 ° C. A solution of the 4″-O-(2,2,2-trichloroethoxycarbonyl)-protected erythromycin derivative (0.386 mmol) in 10 mL of tetrahydrofuran is added, and the mix is stirred for 1 hour at −78 ° C. The reaction is quenched by addition of sat. NaHCO₃, warmed to ambient temperature, and extracted with ether. The extract is dried over MgSO₄, filtered, and evaporated. The product is purified by silica gel chromatography.

### EXAMPLE 16

### 12-membered macrolide 12,13-epoxide

A solution of the 12-membered macrolide (1 mmol; Example 5) in CH₂Cl₂ is added to a solution of bis[α,α-bis(trifluoromethyl)benzenemethanolato]-diphenylsulfur (1.5 g) in CH₂Cl₂. After 45 minutes, a second portion of sulfurane (0.75 g) is added, and the reaction is continued for an additional 30 minutes. The mixture is poured into ethyl acetate and 5% aqueous NaHCO₃ is added until the pH of the aqueous phase reaches 7. The organic phase is separated, and the aqueous phase is extracted three times with ethyl acetate. The organic solutions are combined, washed with aq. NaCl, dried over MgSO₄, filtered, and evaporated. The product is isolated by silica gel chromatography.

### EXAMPLE 17

### Erythromycin A Lactams

A solution of the epoxide (1 mmol; Example 16) and ammonium chloride (2 g) in 7 N methanolic ammonia (100 mL) is placed in a sealed bomb and heated at 100 ° C. for 4 days. The bomb is cooled and opened, and the mixture is evaporated to dryness. The product is isolated by silica gel chromatography.

### EXAMPLE 18

### N-alkyl-Erythromycin A Lactams

A solution of the epoxide (1 mmol; Example 16), the alkylamine R-NH₂ (0.5 mol), and conc. HCl (5 mmol) is placed in a sealed bomb and heated at 100° C. for 4 days. The bomb is cooled and opened, and the mixture is evaporated to dryness. The product is isolated by silica gel chromatography.

### EXAMPLE 19

### 3′-N-desmethyl-erythromycin Lactam

9-deoxo-6,9-epoxy-erythromycin A lactam (1 mmol; Example 17) and sodium acetate trihydrate (690 mg) in 10 mL of 80:20 methanol/water is heated to 47° C. and treated with iodine (257 mg). The pH is maintained in the range of 8-9 by addition of 1 N NaOH when needed. After 2 hours, the colorless mixture is poured into water and adjusted to pH 10, then extracted with CH₂Cl₂. The extract is washed sequentially with aq. Na₂S₂O₃ and brine, then dried over MgSO₄, filtered, and evaporated. The product is isolated by silica gel chromatography.

### EXAMPLE 20

### Alternate Preparation of 3′-N-desmethyl-erythromycin Lactam

A solution of-deoxo-6,9-epoxy-13-erythromycin A lactam (1 mmol; Example 17) in 40 mL of anhydrous CH₃CN at 0° C. is treated with N-iodosuccinimide (270 mg) in small portions. After addition, the mixture is kept at ambient temperature for 12 hours. The mixture is diluted with ethyl acetate and washed sequentially with aq. NaHSO₃, 5% Na₂CO₃, and brine, then dried over MgSO₄, filtered, and evaporated. The product is isolated by silica gel chromatography.

### EXAMPLE 21

### 3′-N-desmethyl-3′-N-alkyl-erythromycin Lactam

A solution of the 3′-N-desmethyl-9-deoxo-6,9-epoxy-erythromycin A lactam (1 mmol; Examples 19 and 20) in CH₃CN (10 mL) is treated with solid NaHCO₃ (420 mg) and an alkylating agent (e.g., alkyl halide or alkyl sulfonate) (1.1 mmol) with stirring for 2 days at ambient temperature. The mixture is diluted with ethyl acetate and washed sequentially with sat. NaHCO₃ and brine, then dried with MgSO₄, filtered, and evaporated. The product is isolated by silica gel chromatography.

### EXAMPLE 22

### Alternate Preparation of 3′-N-desmethyl-3′-N-alkyl-erythromycin Lactam

A solution of the 3′-N-desmethyl-9-deoxo-6,9-epoxy-erythromycin A lactam (2 mmol; Examples 19 and 20) and an aldehyde or ketone (8 mmol) in 20 mL of methanol is treated with acetic acid (0.46 mL) and sodium cyanoborohydride (0.25 g) for 12 hours at ambient temperature. Additional aldehyde or ketone (4 mmol), acetic acid (0.23 mL), and NaBH₃CN (0.13 g) is added and the reaction is allowed to continue an additional 24 hours. The mixture is concentrated to dryness, then dissolved in CH₂Cl₂ and washed sequentially with 5% aq. Na₂CO₃ and brine, dried over MgSO₄, filtered, and evaporated. The product is isolated by silica gel chromatography.

### EXAMPLE 23

### 2′-O-Acetyl-15-methylerythromycin A

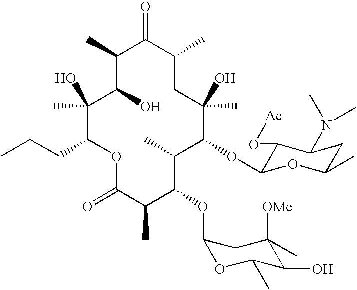

A solution of acetic anhydride (1.39 mL) in 2 mL of ethyl acetate was added to a solution of 15-methylerythromycin A (10.0 g, ˜90% pure) in 50 mL of ethyl acetate at 0° C. and the mixture was stirred for 30 minutes, then warmed to ambient temperature and stirred for 4 hours. The mixture was treated with sat. NaHCO₃ for 30 minutes, then extracted three times with ethyl acetate. The extract was dried over MgSO₄, filtered, and evaporated. The product was crystallized from CH₂Cl₂ and hexane, yielding 7.5 g of product.

### EXAMPLE 24

### 2′-O-Acetyl-4″-O-(imidazolylthiocarbonyl)-15-methylerythromycin A

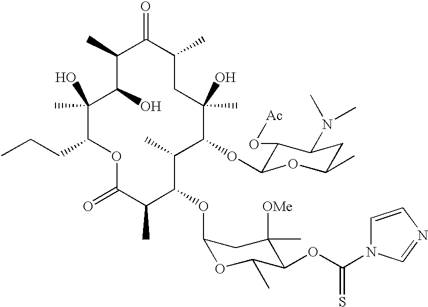

A mixture of 2′-O-acetyl-15-methylerythromycin A (4.5 g), 1,1′-thiocarbonyldiimidazole (1.52 g), and 4-(dimethylamino)pyridine (1.04 g) in CH₂Cl₂ (50 mL) was stirred for 12 hours at ambient temperature. Additional portions of 1,1′-thiocarbonyldiimidazole (1.0 g), and 4-(dimethylamino)pyridine (0.68 g) were added and the reaction was continued an additional 24 hours. The reaction was warmed until complete disappearance of starting material as evidenced by thin-layer chromatography (1:1 acetone/hexane, pretreating plate with NH₃ vapor). The mixture was diluted with CH₂Cl₂ and washed sequentially with sat. NaHCO₃ and water, then dried over MgSO₄, filtered, and evaporated. The crude product was partially purified by flash chromatography on silica gel (gradient of 2:1 to 1:1 hexanes/acetone+1% Et₃N) to provide 4.5 g of light yellow solid.

### EXAMPLE 25

### 2′-O-Acetyl-4″-deoxy-15-methylerythromycin A

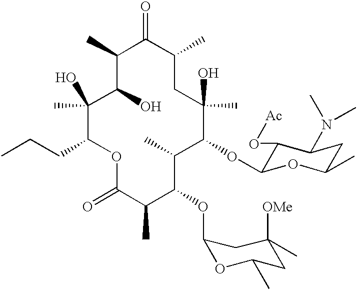

A solution of 2′-O-acetyl-4″-O-(imidazolylthiocarbonyl)-15-methylerythromycin A (1.0 g) in 25 mL of toluene under inert atmosphere was warmed in a 100° C. oil bath and treated with 1,1′-azobis(cyclohexanecarbonitrile) (50 mg) followed by dropwise addition of tri-n-butyltin hydride (0.9 mL). Heating is continued for 1 hour, at which time thin-layer chromatographic analysis indicated completion of reaction. The reaction was repeated at 3.5-times the scale, and the final solutions were combined and evaporated. The residue was dissolved in 600 mL of acetonitrile and washed with hexanes (5×200 mL). The hexanes washes were combined and extracted once with acetonitrile. The acetonitrile solutions were combined and evaporated to yield crude product, which was purified by silica gel chromatography (2:1 hexane/acetone+1% Et₃N) to give 3.1 g of product.

### EXAMPLE 26

### 2′-O-Acetyl-4″-deoxy-15-methylerythromycin A 11,12-cyclic Carbonate

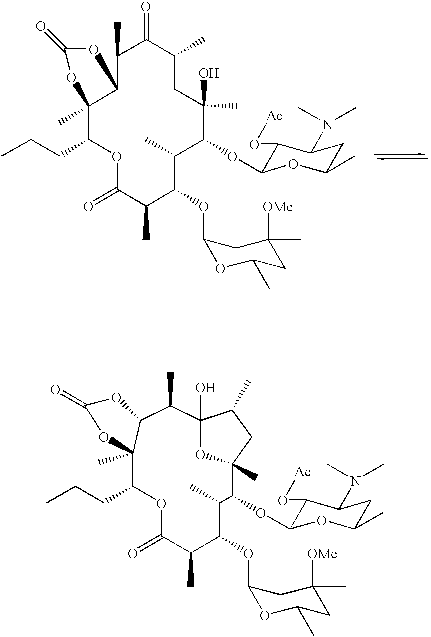

A solution of 2′-O-acetyl-4″-deoxy-15-methylerythromycin A (3.1 g), 1,1-carbonyldiimidazole (2.6 g), and 4-(dimethylamino)pyridine (0.98 g) in 20 mL of toluene was heated at 80° C. for 1.5 hours. The reaction was cooled, diluted with CH₂Cl₂, and washed sequentially with cold buffer (5% KH₂PO₄+1% K₂HPO₄) and sat. NaHCO₃. The solution was dried over MgSO₄, filtered, and evaporated. The crude product was purified by flash chromatography on silica gel (hexanes/acetone+1% Et₃N) to provide 2.3 g of white solid.

### EXAMPLE 27

### (9S)-2′-O-Acetyl-4″-deoxy-9-dihydro-15-methylerythromycin A 11,12-cyclic Carbonate

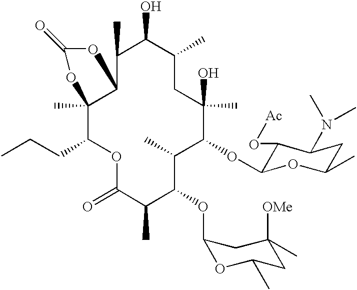

A solution of 2′-O-acetyl-4″-deoxy-15-methylerythromycin A 11,12-cyclic carbonate (1.47 g) in 2-propanol (15 mL) and ether (45 mL) was treated with sodium borohydride (85 mg) at ambient temperature. After 4 hours, an additional 85 mg of sodium borohydride was added and the mixture was stirred 12 hours. The mixture was diluted with cold buffer (5% KH₂PO₄+1% K₂HPO₄) and extracted with CH₂Cl₂. The extract was washed with buffer then dried over MgSO₄, filtered, and evaporated. The crude product was purified by flash chromatography on silica gel (2:1 hexanes/acetone+1% Et₃N) to provide 0.8 g of white solid. NMR analysis indicated this material to be ˜85% pure and to contain ˜15% material lacking the 11,12-cyclic carbonate.

### EXAMPLE 28

### (8R,9R)-2′-O-Acetyl-4″-deoxy-9-deoxo-6,9-epoxy-15-methylerythromycin A 11,12-cyclic Carbonate

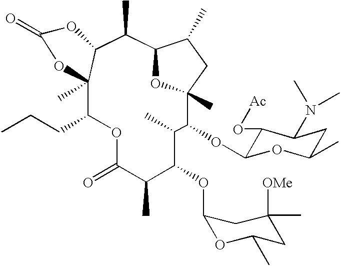

A solution of (9S)-2′-O-acetyl-4″-deoxy-9-dihydro-15-methylerythromycin A 11,12-cyclic carbonate (798 mg) in 30 mL of CH₂Cl₂ was cooled on ice and treated with pyridine (253 uL) followed by trifluoromethanesulfonic anhydride (336 uL). After 30 minutes, sat. NaHCO3 was added and the mixture was extracted with CH₂Cl₂. The extract was washed with water, then dried over MgSO₄, filtered, and evaporated. The crude product was purified by flash chromatography on silica gel (2:1 hexanes/acetone+1% Et₃N) to provide 600 mg of solid.

### EXAMPLE 29

### Ring-contracted (8R,9R)-4″-deoxy-9-deoxo-6,9-epoxy-15-methylerythromycin A

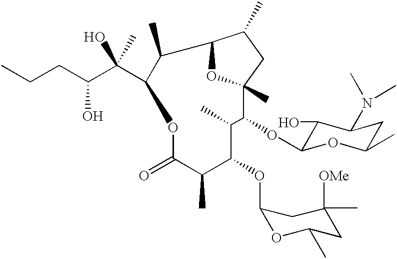

A solution of (8R,9R)-2′-O-acetyl-4″-deoxy-9-deoxo-6,9-epoxy-15-methylerythromycin A 11,12-cyclic carbonate (600 mg) and potassium carbonate (317 mg) in 15 mL of anhydrous methanol was stirred at ambient temperature for 3 hours, then heated in a 70° C. oil bath for an additional 8 hours. The solvent was evaporated, and the residue was partitioned between ethyl acetate and sat. NaHCO₃. The ethyl acetate extract was dried over MgSO₄, filtered, and evaporated. The crude product was purified by flash chromatography on silica gel (1:1 hexanes/acetone+1% Et₃N) to provide 367 mg.

### EXAMPLE 30

### Ring-contracted (8R,9R)-4″,13-dideoxy-9-deoxo-6,9;12,13-bisepoxy-15-methylerythromycin A

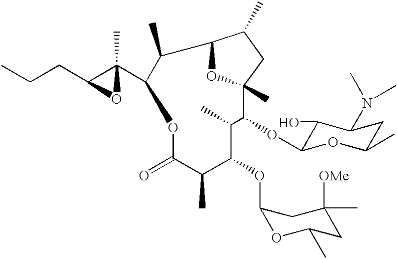

A solution of ring-contracted (8R,9R)-4″-deoxy-9-deoxo-6,9-epoxy-15-methylerythromycin A (367 mg) and the Martin sulfurane (740 mg) in 3 mL of CH₂Cl₂ was stirred at ambient temperature for 45 minutes. The mixture was treated with sat. NaHCO₃ and extracted with ethyl acetate. The ethyl acetate extract was dried over MgSO₄, filtered, and evaporated. The crude product was purified by flash chromatography on silica gel (1:1 hexanes/acetone+1% Et₃N) to provide 326 mg of material containing some diphenylsulfide impurity.

### EXAMPLE 31

### (8R,9R)-4″-deoxy-9-deoxo-6,9-epoxy-15-methylerythromycin A Lactam

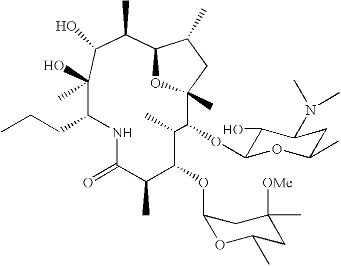

A solution of ring-contracted (8R,9R)-4″,13-dideoxy-9-deoxo-6,9;12,13-bisepoxy-15-methylerythromycin (250 mg) and ammonium chloride (178 mg) in 10 mL of 7 M NH₃ in methanol was sealed in a bomb and heated in a 120° C. oil bath for 4.5 days. The bomb was cooled and opened, and the mixture was diluted with water and ethyl acetate. The phases were partitioned, and the water was extracted 3× with ethyl acetate. The organic phases were combined, dried over MgSO₄, filtered, and evaporated. The crude product was purified by flash chromatography on silica gel (1:1 hexanes/acetone+1% Et₃N) to provide 866 mg of white solid.

### EXAMPLE 32

### (8R,9R)-N-desmethyl-4″-deoxy-9-deoxo-6,9-epoxy-15-methylerythromycin A Lactam

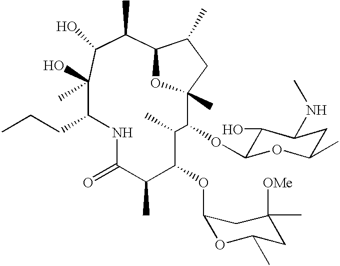

A solution of (8R,9R)-4″-deoxy-9-deoxo-6,9-epoxy-15-methylerythromycin A lactam (80 mg), sodium acetate (46 mg), and iodine (28.5 mg) in 10 mL of 80:20 methanol/water was heated at 50° C. and 0.6 mL of 0.2 M LiOH was added in portions to keep the pH between 8 and 9. After 2 hours, the colorless solution was poured into water, adjusted to pH 10, and extracted with CH₂Cl₂. The extract was washed sequentially with 5% Na₂S₂O₃ and brine, then dried over MgSO₄, filtered, and evaporated. The product (82 mg) was used without further purification.

### EXAMPLE 33

### (8R,9R)-N-desmethyl-N-isopropyl-4″-deoxy-9-deoxo-6,9-epoxy-15-methylerythromycin A Lactam

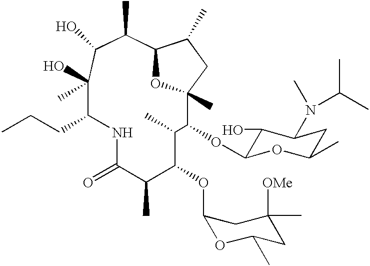

A solution of (8R,9R)-N-desmethyl-4″-deoxy-9-deoxo-6,9-epoxy-15-methyl-erythromycin A lactam (82 mg), 2-iodopropane (402 uL), and diisopropylethylamine (200 uL) in 4 mL of acetonitrile was heated in a 70° C. bath for 24 hours. The mixture was treated with water and sat. NaHCO₃ and extracted with ethyl acetate. The extract was dried over MgSO₄, filtered, and evaporated. The crude product was purified by flash chromatography on silica gel (2:1 hexanes/acetone+1% Et₃N) to provide 37 mg of white solid. ¹³C-NNM (d₆-acetone): □178.8; 104.6; 96.7; 89.5; 85.0; 84.5; 79.6; 76.6; 72.1; 71.9; 69.7; 64.0; 62.4; 56.6; 54.2; 50.5; 48.4; 47.7; 44.1; 44.0; 35.0; 34.6; 34.5; 32.2; 32.0; 29.3; 26.8; 23.2; 22.6; 22.3; 21.6; 21.5; 18.7; 16.3; 15.8; 15.2; 10.4; 10.2.

### EXAMPLE 34

### 2′-O-Acetyl-4″-O-(2,2,2-trichloroethoxycarbonyl)-15-methylerythromycin A

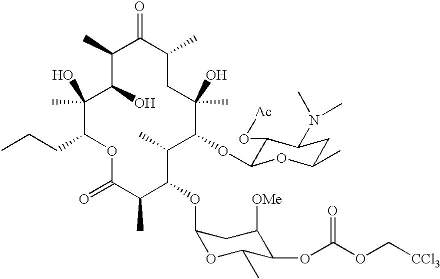

Trichloroethyl chloroformate (2.5 g) is added dropwise to a mixture of 2′-O-acetyl-15-methylerythromycin A (7.9 g) and 4-(dimethylamino)pyridine (1.5 g) in CH₂Cl₂ (100 mL) at −78° C., and the mixture is stirred for 24 hours. After warming to ambient temperature, the mixture is diluted with CH₂Cl₂ and washed sequentially with phsphate buffer (5% KH₂PO₄+1% K₂HPO₄) and brine, then dried over MgSO₄, filtered, and evaporated. The crude product is purified by flash chromatography on silica.

### EXAMPLE 35

### 2′-O-Acetyl-4″-O-(2,2,2-trichloroethoxycarbonyl)-15-methylerythromycin A 11,12-cyclic Carbonate

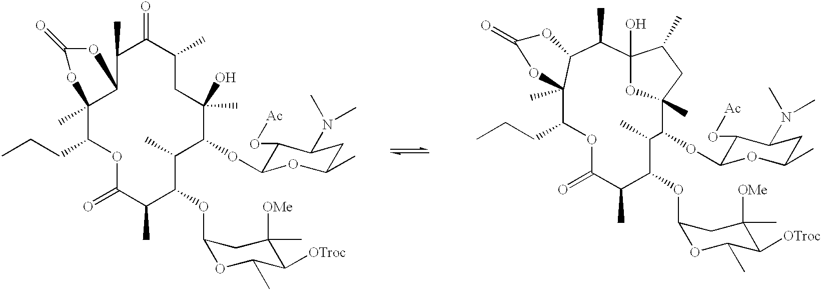

A solution of 2′-O-acetyl-4″-O-(2,2,2-trichloroethoxycarbonyl)-15-methylerythromycin A (3.2 g), 1,1-carbonyldiimidazole (2.6 g), and 4-(dimethylamino)pyridine (0.98 g) in 20 mL of toluene is heated at 80° C. for 1.5 hours. The reaction is cooled, diluted with CH₂Cl₂, and washed sequentially with cold buffer (5% KH₂PO₄+1% K₂HPO₄) and sat. NaHCO₃. The solution is dried over MgSO₄, filtered, and evaporated. The crude product is purified by flash chromatography on silica gel (hexanes/acetone+1% Et₃N).

### EXAMPLE 36

### (9S)-2′-O-Acetyl-4″-O-(2,2,2-trichloroethoxycarbonyl)-9-dihydro-15-methylerythromycin A 11,12-cyclic Carbonate

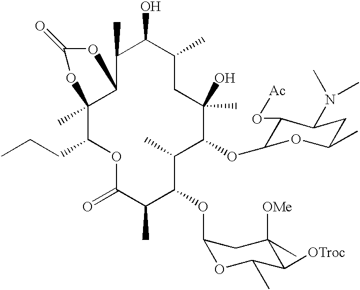

A solution of 2′-O-acetyl-4″-O-(2,2,2-trichloroethoxycarbonyl)-15-methylerythromycin A 11,12-cyclic carbonate (1.8 g) in 2-propanol (15 mL) and ether (45 mL) is treated with sodium borohydride (85 mg) at ambient temperature. After 4 hours, an additional 85 mg of sodium borohydride is added and the mixture is stirred 12 hours. The mixture is diluted with cold buffer (5% KH₂PO₄+1% K₂HPO₄) and extracted with CH₂Cl₂. The extract is washed with buffer then dried over MgSO₄, filtered, and evaporated. The crude product is purified by flash chromatography on silica gel (hexanes/acetone+1% Et₃N).

### EXAMPLE 37

### (8R,9R)-2′-O-Acetyl-4″-O-(2,2,2-trichloroethoxycarbonyl)-9-deoxo-6,9-epoxy-15-methylerythromycin A 11,12-cyclic Carbonate

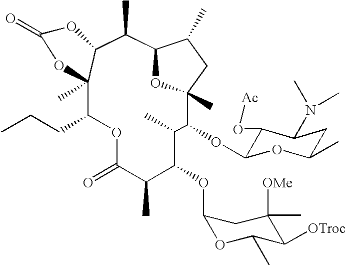

A solution of (9S)-2′-O-acetyl-4″-O-(2,2,2-trichloroethoxycarbonyl)-9-dihydro-15-methylerythromycin A 11,12-cyclic carbonate (990 mg) in 30 mL of CH₂Cl₂ is cooled on ice and treated with pyridine (253 uL) followed by trifluoromethanesulfonic anhydride (336 uL). After 30 minutes, sat. NaHCO3 is added and the mixture is extracted with CH₂Cl₂. The extract is washed with water, then dried over MgSO₄, filtered, and evaporated. The crude product is purified by flash chromatography on silica gel (hexanes/acetone+1% Et₃N.

### EXAMPLE 38

### Ring-contracted (8R,9R)-9-deoxo-6,9-epoxy-15-methylerythromycin A

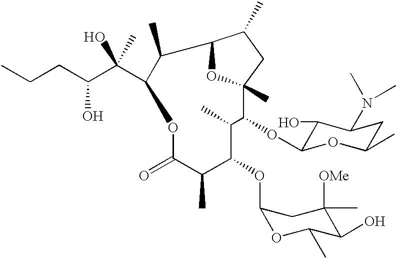

A solution of (8R,9R)-2′-O-acetyl-4″-O-(2,2,2-trichloroethoxycarbonyl)-9-deoxo-6,9-epoxy-15-methylerythromycin A 11,12-cyclic carbonate (750 mg) and potassium carbonate (317 mg) in 15 mL of anhydrous methanol is stirred at ambient temperature for 3 hours, then heated in a 70° C. oil bath for an additional 8 hours. The solvent is evaporated, and the residue is partitioned between ethyl acetate and sat. NaHCO₃. The ethyl acetate extract is dried over MgSO₄, filtered, and evaporated. The crude product is purified by flash chromatography on silica gel (hexanes/acetone+1% Et₃N).

### EXAMPLE 39

### Ring-contracted (8R,9R)-13-deoxy-9-deoxo-6,9;12,13-bisepoxy-15-methylerythromycin A

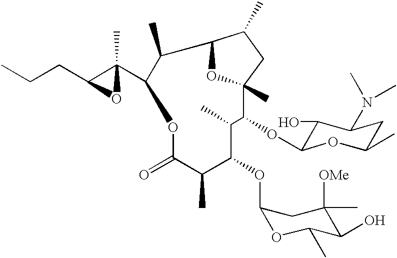

A solution of ring-contracted (8R,9R)-9-deoxo-6,9-epoxy-15-methylerythromycin A (450 mg) and the Martin sulfurane (740 mg) in 3 mL of CH₂Cl₂ is stirred at ambient temperature for 45 minutes. The mixture is treated with sat. NaHCO₃ and extracted with ethyl acetate. The ethyl acetate extract is dried over MgSO₄, filtered, and evaporated. The crude product is purified by flash chromatography on silica (hexanes/acetone+1% Et₃N).

### EXAMPLE 40

### (8R,9R)-9-deoxo-6,9-epoxy-15-methylerythromycin A Lactam

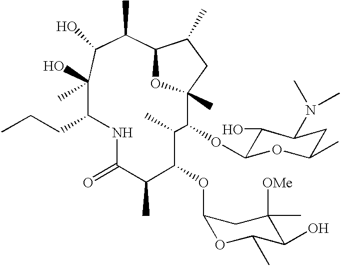

A solution of ring-contracted (8R,9R)-13-deoxy-9-deoxo-6,9;12,13-bisepoxy-15-methylerythromycin (310 mg) and ammonium chloride (178 mg) in 10 mL of 7 M NH₃ in methanol is sealed in a bomb and heated in a 120° C. oil bath for 4.5 days. The bomb is cooled and opened, and the mixture is diluted with water and ethyl acetate. The phases are partitioned, and the water is extracted 3× with ethyl acetate. The organic phases are combined, dried over MgSO₄, filtered, and evaporated. The crude product is purified by flash chromatography on silica gel (hexanes/acetone+1% Et₃N).

### EXAMPLE 41

### (8R,9R)-N-desmethyl-9-deoxo-6,9-epoxy-15-methylerythromycin A Lactam

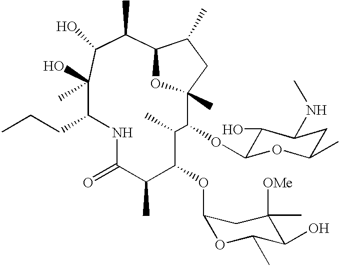

A solution of (8R,9R)-9-deoxo-6,9-epoxy-15-methylerythromycin A lactam (100 mg), sodium acetate (46 mg), and iodine (28.5 mg) in 10 mL of 80:20 methanol/water is heated at 50° C. and 0.6 mL of 0.2 M LiOH is added in portions to keep the pH between 8 and 9. After 2 hours, the colorless solution is poured into water, adjusted to pH 10, and extracted with CH₂Cl₂. The extract is washed sequentially with 5% Na₂S₂O₃ and brine, then dried over MgSO₄, filtered, and evaporated. The product is used without further purification.

### EXAMPLE 42

### (8R,9R)-N-desmethyl-N-isopropyl-9-deoxo-6,9-epoxy-15-methylerythromycin A Lactam

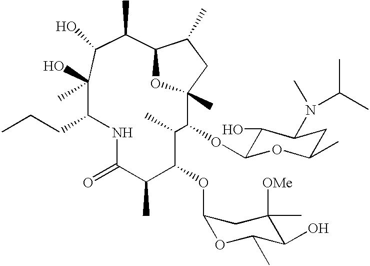

A solution of (8R,9R)-N-desmethyl-9-deoxo-6,9-epoxy-15-methyl-erythromycin A lactam (100 mg), 2-iodopropane (402 uL), and diisopropylethylamine (200 uL) in 4 mL of acetonitrile is heated in a 70° C. bath for 24 hours. The mixture is treated with water and sat. NaHCO₃ and extracted with ethyl acetate. The extract is dried over MgSO₄, filtered, and evaporated. The crude product is purified by flash chromatography on silica gel (hexanes/acetone+1% Et₃N).

### EXAMPLE 43

### Erythromycin A 9-oximino Ether

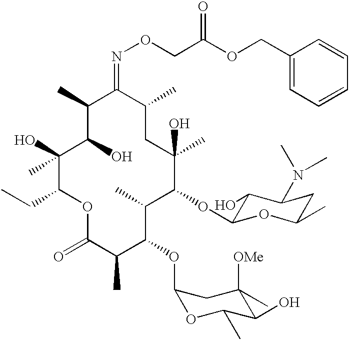

To a suspension of erythromycin A (100 g, 136 mmol) in 2-propanol (200 mL) was added 50% aqueous hydroxylamine (80 mL, 1.36 mol), followed by acetic acid (32.5 mL, 570 mmol). The reaction mixture was heated at 50° C. overnight. A precipitate formed upon cooling to room temperature, which was collected by filtration and washed with water.

Vacuum drying gave a white solid (55.1 g) of the acetate salt of erythromycin A 9-oxime. The combined aqueous filtrate was treated with 4N NaOH(aq) to adjust the pH to above 11, and was extracted with ethyl acetate. The ethyl acetate extract was washed with saturated aqueous sodium bicarbonate and saturated aqueous sodium chloride, dried over anhydrous sodium sulfate. Filtration and solvent evaporation gave a white solid, which was recrystallized from 2-propanol/water, giving another 30.4 g of erythromycin A 9-oxime. ¹H NMR (CDCl₃) δ0.84 (t, 3), 1.05 (d, 3), 1.10 (d, 3), 1.13 (s, 3), 1.18 (d, 3), 1.2-1.3 (m, 2), 1.22 (d, 3), 1.24 (s, 3), 1.28 (d, 3), 1.42-1.68 (m, 5), 1.49 (s, 3), 1.88-2.03 (m, 3), 2.24 (d, 1), 2.29 (s, 6), 2.36 (d, 1), 2.44 (m, 1), 2.69 (q, 1), 2.90 (m, 1), 3.02 (t, 1), 3.13 (br. s, 1), 3.24 (dd, 1), 3.32 (s, 3), 3.52 (m, 1), 3.59 (d, 1), 3.68 (br. s, 1), 3.79 (m, 1), 3.98-4.05 (m, 2), 4.41 (br. s, 1), 4.45 (d, 1), 4.91 (d, 1), 5.09 (dd, 1). MS m/z 750 (M+H⁺).

To a solution of erythromycin A 9-oxime (1.0 g, 1.33 mmol) in DMF (4 mL) cooled in an ice water bath was added benzyl 2-bromoacetate (0.23 mL, 1.5 mmol), followed by 85% KOH powder (0.1 g, 1.5 mmol). The mixture was stirred at 0° C. for 2 h (TLC showed reaction complete). The solution was added to water (100 mL) with stirring. The precipitate formed was collected by filtration, washed thoroughly with water and dried in vacuo. The crude product was re-crystallized from ethyl acetate-hexanes, giving the product as a white powder, 0.79 g. ¹H NMR (CDCl₃) δ0.84 (t, 3), 1.03 (d, 3), 1.08-1.18 (m, 12), 1.2-1.3 (m, 8), 1.35 (d, 3), 1.43-1.68 (m, 6), 1.49 (s, 3), 1.92 (m, 1), 2.06 (t, 1), 2.30 (s, 6), 2.37 (d, 1),2.58 (s, 1), 2.68 (m, 1), 2.91 (m, 1), 3.04 (t, 1), 3.05 (br. s, 1), 3.16 (br. s, 1), 3.24 (dd, 1), 3.32 (s, 3), 3.50 (m, 1), 3.60 (d, 1), 3.76 (m, 1), 3.89 (br. s, 1), 3.94 (s, 1), 4.05 (m, 1), 4.20 (d, 1), 4.42 (d, 1), 4.56 (d, 1), 4.64 (d, 1), 4.93 (d, 1), 5.14 (dd, 1), 5.19 (d, 1), 5.37 (d, 1),. 7.30-7.46 (m, 5). ¹³C NMR (CDCl₃) δ9.2, 10.7, 14.6, 16.2, 18.6, 18.7, 21.2, 21.4, 21.5, 26.6, 26.7, 28.8, 33.2, 35.1, 38.0, 39.0, 40.3, 44.8, 49.5, 65.4, 67.2, 68.8, 70.2, 70.4, 71.1, 72.7, 74.3, 75.0, 78.2, 80.2, 83.7, 96.3, 103.2, 128.5, 128.6, 129.4, 135.5, 170.2, 172.9, 175.1. MS m/z 898 (M+H⁺).

Other compounds were prepared using similar methods where the benzyl 2-bromoacetate was replaced with the appropriate alkyl or aryl halide to yield compounds where the C-9 oxime is ═NOR¹³ wherein R¹³ is

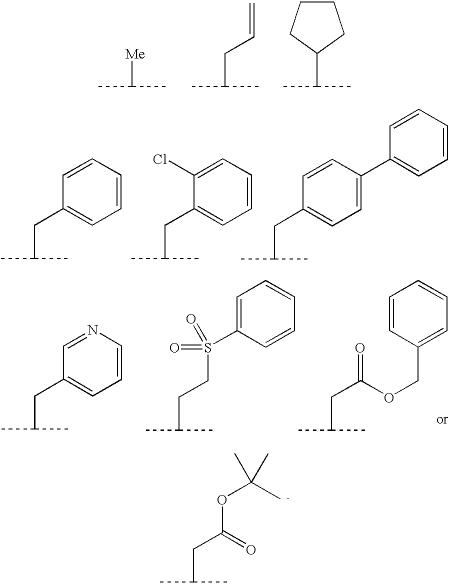

### EXAMPLE 44

### Synthesis of 3′-N-alkyl-3′-N-desmethylerythromycin A 9-oximinoether (3)

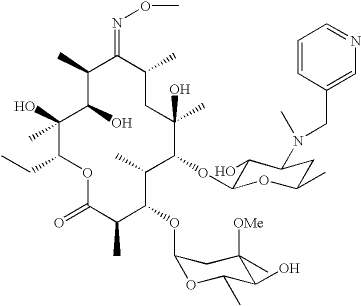

To a solution or suspension of erythromycin A 9-methoxime (3.81 g, 5.0 mmol) in methanol (90 mL) was added a solution of sodium acetate trihydrate (3.4 g, 25.0 mmol) in water (10 mL). The mixture was heated to 50° C. and iodine crystals (1.4 g, 5.5 mmol) were added. Three portions of aqueous NaOH (4 M, 0.5 mL each) were added at 5 min., 15 min., and 1 h. The solution was stirred at 50° C. for 2 h (TLC showed reaction complete) and cooled to room temperature. An aqueous solution of 0.5 M Na₂S₂O₃ (1.0 mL) was added to quench the excess iodine. After addition of 1.5 mL of 4 M NaOH(aq), the solution was added to water (500 mL) with stirring. The precipitate was collected by filtration and washed thoroughly with water. The crude product was recrystallized from ethyl acetate-hexanes to give 3′-N-desmethylerythromycin A 9-methoxime as a white solid, 2.8 g. ¹H NMR (CDCl3) δ0.84 (t, 3), 1.03 (d, 3), 1.04 (d, 3), 1.13 (s, 3), 1.15-1.25 (m, 2), 1.17 (d, 3), 1.18 (d, 3), 1.21 (d, 3), 1.24 (s, 3), 1.29 (d, 3), 1.42-1.59 (m, 4), 1.45 (s, 3), 1.87-2.03 (m, 3), 2.34 (d, 1), 2.41 (s, 3), 2.47 (m, 1), 2.65 (q, 1), 2.88 (m, 1), 3.01 (d, 1), 3.17 (dd, 1), 3.30 (s, 3), 3.54 (m, 1), 3.55 (d, 1), 3.65-3.70 (m, 2), 3.82 (s, 3), 3.98-4.04 (m, 2), 4.37 (d, 1), 4.39 (br. s, 1), 4.91 (d, 1), 5.11 (dd, 1). ¹³C NMR (CDCl₃) δ9.6, 10.7, 14.4, 16.1, 16.3, 18.5, 18.6, 21.1, 21.5, 26.3, 26.8, 32.9, 33.2, 35.0, 37.2, 37.8, 38.9, 44.7, 49.4, 60.3, 61.8, 65.3, 68.6, 70.5, 72.8, 74.2, 74.9, 75.3, 77.9, 80.0, 84.1, 96.3, 102.7, 171.4, 175.0. MS m/z 750 (M+H⁺).

To a solution of 3′-N-desmethylerythromycin A 9-methoxime (1.5 g, 2 mmol) in MeOH (20 mL) was added 3-pyridinecarboxyaldehyde (0.76 mL, 8 mmol), acetic acid (1.0 mL, 18 mmol), and NaBH₃CN (0.5 g, 8 mmol). The mixture was heated to 50° C. for 2 h (TLC showed reaction complete). The mixture was treated with 20 mL of 1M aq. K₂CO₃, concentrated on a rotary evaporator, and extracted with EtOAc. The EtOAc was washed with aq NaHCO₃ and brine, and dried over Na₂SO₄. The crude solid obtained was recrystallized from EtOAc/Hexane, giving a white solid, 1.3 g (77% yield). ¹H NMR (CDCl3) δ0.84 (t, 3), 1.02 (d, 3), 1.09 (d, 3), 1.13 (s, 3), 1.17 (d, 3), 1.18 (d, 3), 1.2-1.3 (m, 2), 1.22 (s, 3), 1.25 (d, 3), 1.27 (d, 3), 1.4-1.6 (m, 4), 1.46 (s, 3), 1.76 (m, 1), 1.87-2.06 (m, 3), 2.24 (s, 3), 2.33 (d, 1), 2.57 (m, 1), 2.65 (q, 1), 2.89 (m, 1), 3.01 (d, 1), 3.20 (s, 3), 3.36 (dd, 1), 3.46-3.53 (m, 2), 3.57 (d, 1), 3.65-3.70 (m, 2), 3.78 (d, 1), 3.81 (s, 3), 3.98-4.03 (m, 2), 4.39 (br. s, 1), 4.41 (d, 1), 4.74 (s, 1), 4.91 (d, 1), 5.11 (dd, 1) 7.26 (m, 1), 7.65 (dt, 1), 8.50-8.53 (m, 2). ¹³C NMR (CDCl₃) δ9.2, 10.7, 14.5, 16.1, 16.2, 18.5, 18.6, 21.1, 21.4, 21.5, 26.3, 27.0, 30.2, 32.9, 35.0, 36.9, 37.8, 39.0, 44.7, 49.4, 55.3, 61.8, 62.7, 64.5, 65.5, 68.7, 70.5, 71.1, 72.7, 74.3, 75.3, 78.0, 79.8, 83.3, 96.2, 102.8, 123.5, 134.4, 136.4, 148.7, 150.2, 171.5, 175.2. HRMS m/z calculated for C₄₃H₇₄N₃O₁₃ (M+H⁺) 840.5216, observed 840.5184.

The oximes of Example 43 were further modifed at the desosamine nitrogen using a similar methods except that 3-pyridinecarboxyaldehyde was replaced with the appropriate aldehyde to yield compounds where R² is methyl and R³ is

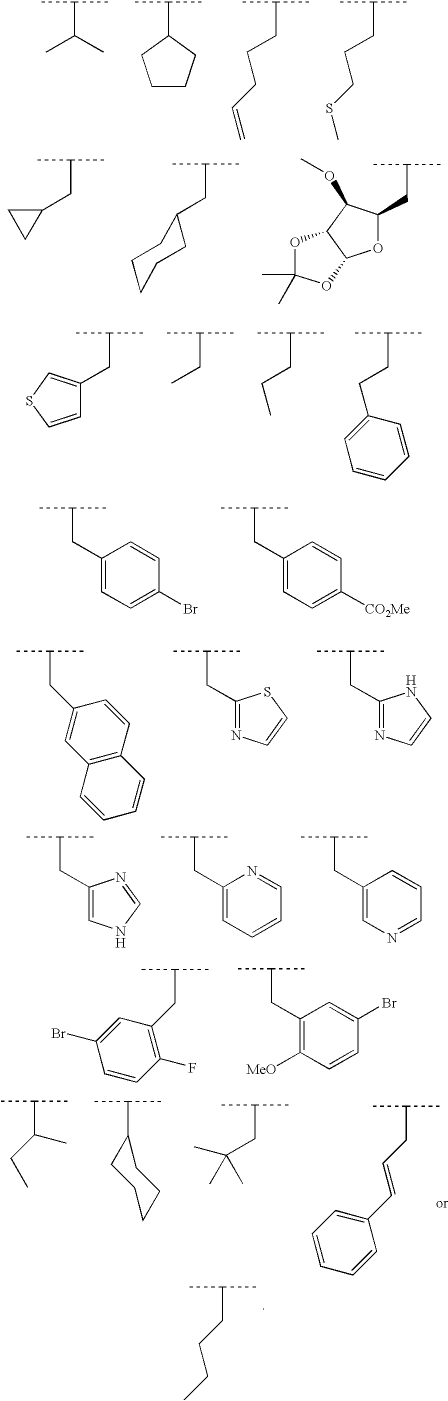

### EXAMPLE 45

### 4″-O-acyl-3′-N-alkyl-3′-N-desmethylerythromycin A 9-oximinoether

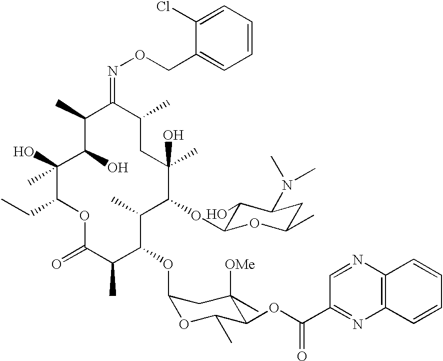

To a solution of erythromycin A 9-(2-chlorobenzyl)oxime (1.74 g, 2 mmol) in ethyl acetate (15 mL) was added acetic anhydride (0.38 mL, 4 mmol). The mixture was stirred at room temperature overnight (TLC showed reaction complete). After stirring with aqueous sodium bicarbonate for half an hour, the mixture was extracted with ethyl acetate. The ethyl acetate solution was washed with saturated aqueous sodium bicarbonate and saturated aqueous sodium chloride, dried over anhydrous sodium sulfate. Filtration and solvent evaporation gave 1.8 g of 2′-O-acetylerythromycin A 9-(2-chlorobenzyl)oxime as a white solid. MS m/z 916 (M+H⁺).

To a solution of 2′-O-acetylerythromycin A 9-(2-chlorobenzyl)oxime (92 mg, 0.1 mmol) in pyridine (3 mL) was added quinoxaloyl chloride (39 mg, 0.2 mmol) and 4-(dimethylamino)pyridine (25 mg, 0.2 mmol). After the mixture was heated to 80° C. for 2 h, additional quinoxaloyl chloride (39 mg, 0.2 mmol) and 4-(dimethylamino)pyridine (25 mg, 0.2 mmol) was added. The mixture was heated at 80° C. overnight. Pyridine was evaporated and the residue was re-dissolved in dichloromethane. A polystyrene-bound ethylenediamine resin was used to scavenge the excess acyl chloride. The crude product (4″-O-quinoxaloyl-2′-O-acetylerythromycin A 9-(2-chlorobenzyl)oxime) was stirred with methanol overnight to remove the 2′-acetyl group. The 4″-O-quinoxaloylerythromycin A 9-(2-chlorobenzyl)oxime product was purified by HPLC. MS m/z 1031 (M+H⁺). Other oximes of Examples 43 and 44 were further modified at the 4″ hydroxyl of the cladinose using similar methods except that quinoxaloyl chloride was replaced with the appropriate halide to yield compounds where the 4″ hydroxyl has been modified to an —OR¹⁴ wherein R¹⁴ is

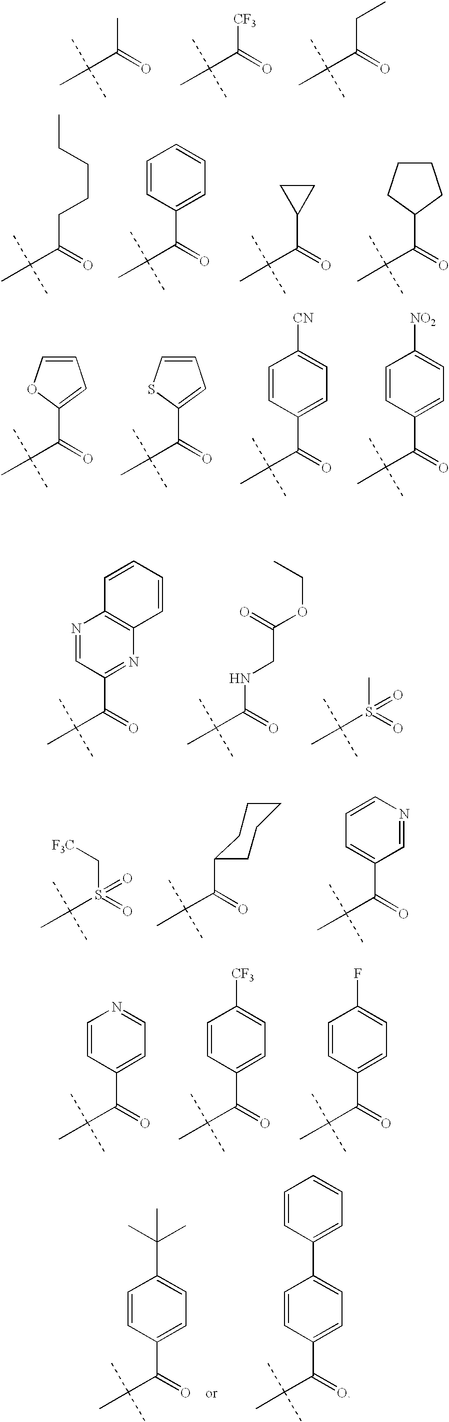

### EXAMPLE 46

### Motilin Receptor Competitive Binding Assay

The compounds of the present invention are tested using a motilin receptor competitive binding assay. An illustrative protocol for such assay is described by Bormans et al, Regul.

Peptides, 15: 143 (1986) which is incorporated herein by reference. In general, membranes prepared from rabbit antrum or duodenum are incubated with 25-50 pM ¹²⁵I-labelled motilin and varying concentration of a test ligand. Protein-bound radioactivity is estimated from parallel reactions to which 100 nm unlabelled motilin is added. Efficacy of a test compound is expressed as IC₅₀, the concentration of the test compound to reduce the specific binding capacity to 50%.

### EXAMPLE 47

### Contractile Activity Assay

The compounds of the present invention are tested in a contractile activity assay described by Depoortere et al, Peptides, 11:515-519 (1990) which is incorporated herein by reference. Briefly, integral segments of rabbit small intestine (1.5-2 cm) are vertically suspended in tissue baths (10 ml), continuously gassed with 95% oxygen, 5% carbon dioxide, and kept at 37° C. The tissue baths contain Hepes buffer (pH 7.4) comprising 137 mM NaCl; 5.9 mM KCl; 1.2 mM CaCl₂; 1.2 mM MgCl₂; 11.6 mM Hepes; and 11.5 mM glucose. Contractions are recorded isotonically. Cumulative concentrations response curves are established by adding logarithmically increasing doses of the test compounds in 100 μl quantities to the bath. From each curve, the negative logarithm of the concentration necessary to induce 50% of the maximal contraction (pED₅₀) is determined by fitting a sigmoid curve to the data.

What is claimed is:

1. A compound of the structure 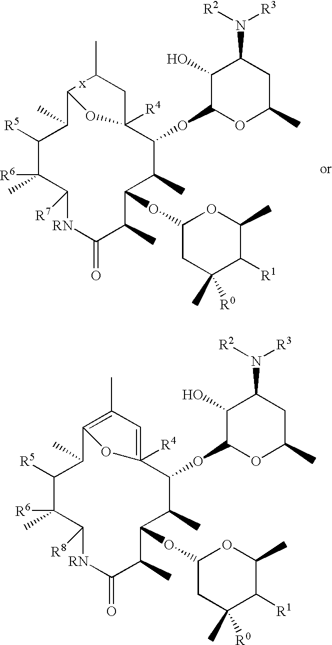
wherein:
R is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;
R⁰ is hydroxyl or methoxy;
R¹ is selected from the group consisting of hydrogen, hydroxyl, halide, NH₂, OR⁹, 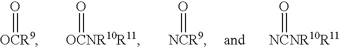
where
R⁹ is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl, and R¹⁰ and R¹¹ are each independently hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;
R² and R³ are each independently selected from the group consisting of hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, and unsubstituted alkynylaryl, or R² and R³ together form a cycloalkyl or an aryl moiety;
R⁴ is hydrogen or methyl;
R⁵ is hydroxyl or oxo;
R⁶ is hydrogen, hydroxyl, or OR¹² where R¹² is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, or unsubstituted C₂-C₁₀ alkynyl;
R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;
R⁸ is unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl; and,
x is a single or a double bond.

2. The compound as in claim 1 wherein:
R is hydrogen, methyl, ethyl, propyl, isopropyl, phenyl or benzyl; R⁰ is hydroxyl or methoxy;
R¹ is hydrogen or hydroxyl;
R² is methyl;
R³ is methyl, ethyl, propyl, isopropyl, butyl, isobutyl, secbutyl or tertbutyl;
R⁴ is methyl;
R⁵ is hydroxyl;
R⁶ is hydroxyl or methoxy;
R⁷ is methyl, vinyl, propyl, isobutyl, pentyl, prop-2-enyl, propargyl, but-3-enyl, 2-azidoethyl, 2-fluoroethyl, 2-chloroethyl, cyclohexyl, phenyl, or benzyl;
R⁸ is methyl, ethyl vinyl, propyl, isobutyl, pentyl, prop-2-enyl, propargyl, but-3-enyl, 2-azidoethyl, 2-fluoroethyl, 2-chloroethyl, cyclohexyl, phenyl, or benzyl; and,
x is a single or a double bond.

3. The compound as in claim 1 of the formula 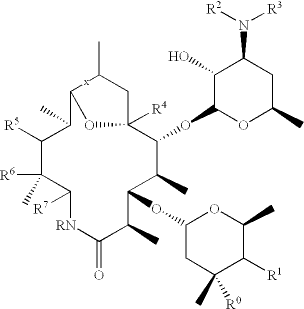
wherein
R is hydrogen, substituted C₁-C₅ alkyl, unsubstituted C₁-C₅ alkyl, substituted aryl, unsubstituted aryl, substituted alkylaryl or unsubstituted alkylaryl;
R⁰ is hydroxyl or methoxy;
R¹ is hydrogen or hydroxyl;
R² and R³ are each independently substituted C₁-C₅ alkyl, unsubstituted C₁-C₅ alkyl, phenyl or benzyl;
R⁴ is methyl;
R⁵ is hydroxyl or oxo;
R⁶ is hydrogen, hydroxyl, or OR¹² wherein R¹² substituted C₁-C₅ alkyl, or unsubstituted C₁-C₅ alkyl;
R⁷ is methyl, unsubstituted C₃-C₅ alkyl, substituted C₂-C₅ alkyl, substituted C₂-C₅ alkenyl, unsubstituted C₂-C₅ alkenyl, substituted C₂-C₅ alkynyl, unsubstituted C₂-C₅ alkynyl, substituted aryl, unsubstituted aryl, substituted alkylaryl unsubstituted alkylaryl, substituted alkenylaryl or unsubstituted alkenylaryl alkenylaryl; and,
x is single bond or a double bond.

4. The compound as in claim 3 wherein x is a single bond.

5. The compound as in claim 1 of the formula 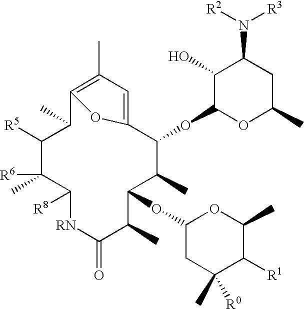
wherein
R is hydrogen, substituted C₁-C₅ alkyl, unsubstituted C₁-C₅ alkyl, substituted aryl, unsubstituted aryl, substituted alkylaryl or unsubstituted alkylaryl;
R⁰ is hydroxyl or methoxy;
R¹ is hydrogen or hydroxyl;
R² and R³ are each independently substituted C₁-C₅ alkyl, unsubstituted C₁-C₅ alkyl, phenyl or benzyl;
R⁵ is hydroxyl or oxo;
R⁶ is hydrogen, hydroxyl, or OR¹² wherein R¹² is substituted C₁-C₅ alkyl, or unsubstituted C₁-C₅ alkyl; and,
R⁸ is substituted C₁-C₅ alkyl, unsubstituted C₁-C₅ alkyl, substituted C₂-C₅ alkenyl, unsubstituted C₂-C₅ alkenyl, substituted C₂-C₅ alkynyl, unsubstituted C₂-C₅ alkynyl, substituted aryl, unsubstituted aryl, substituted alkylaryl unsubstituted alkylaryl, substituted alkenylaryl or unsubstituted alkenylaryl alkenylaryl.

6. A compound of the structure 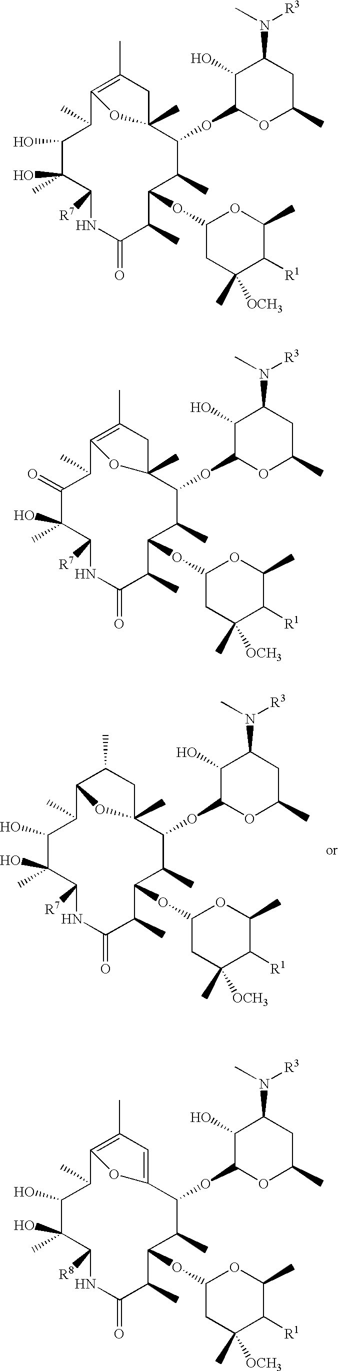
wherein
R is hydrogen, methyl, ethyl, propyl, isopropyl, phenyl or benzyl; R⁰ is hydroxyl or methoxy;
R¹ is hydrogen or hydroxyl;
R³ is methyl, ethyl, propyl, isopropyl, butyl, isobutyl, secbutyl or tertbutyl;
R⁷ is methyl, vinyl, propyl, isobutyl, pentyl, prop-2-enyl, propargyl, but-3-enyl, 2-azidoethyl, 2-fluoroethyl, 2-chloroethyl, cyclohexyl, phenyl, or benzyl;
R⁸ is methyl, ethyl vinyl, propyl, isobutyl, pentyl, prop-2-enyl, propargyl, but-3-enyl, 2-azidoethyl, 2-fluoroethyl, 2-chloroethyl, cyclohexyl, phenyl, or benzyl.

7. The compound as in claim 6 wherein
R³ is methyl, ethyl, or isopropyl;
R⁷ is propyl or fluoroethyl; and
R⁸ is ethyl, propyl or fluoroethyl.

8. The compound as in claim 7 of the structure 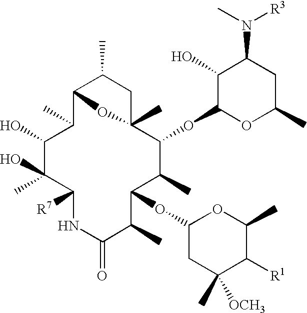
wherein R¹ is hydrogen, R³ is ethyl and R⁷ is propyl.

9. The compound as in claim 7 of the structure 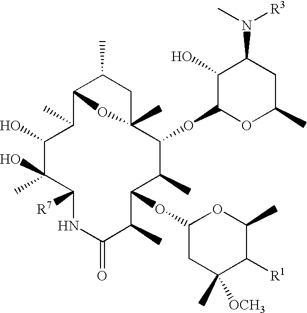
wherein R¹ is hydroxyl, R³ is ethyl and R⁷ is propyl.

10. The compound as in claim 7 of the structure 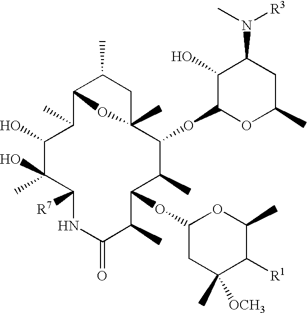
wherein R¹ is hydrogen, R³ is isopropyl and R⁷ is propyl.

11. The compound as in claim 7 of the structure 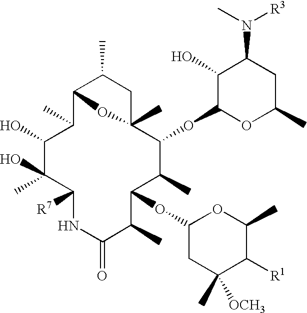
wherein R¹ is hydroxyl, R³ is isopropyl and R⁷ is propyl.

12. The compound as in claim 7 of the structure 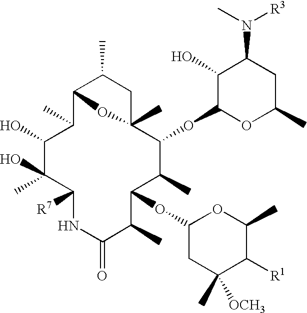
wherein R¹ is hydrogen, R³ is ethyl and R⁷ is fluoroethyl.

13. The compound as in claim 7 of the structure 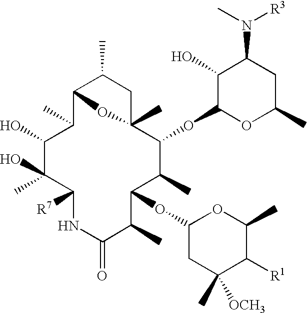
wherein R¹ is hydroxyl, R³ is ethyl and R⁷ is fluoroethyl.

14. The compound as in claim 7 of the structure 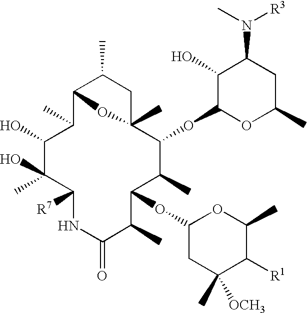
wherein R¹ is hydrogen, R³ is isopropyl and R⁷ is fluoroethyl.

15. The compound as in claim 7 of the structure 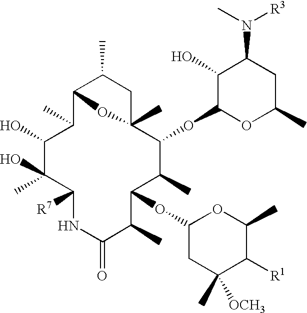
wherein R¹ is hydroxyl, R³ is isopropyl and R⁷ is fluoroethyl.

16. A compound of the structure 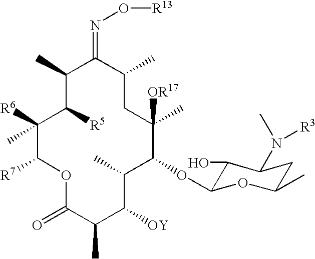
wherein
Y is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, unsubstituted alkynylaryl, unsubstituted cladinose, or substituted cladinose;
R³ is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;
R⁵ is hydroxyl or oxo;
R⁶ is hydrogen, hydroxyl, or OR¹² where R¹² is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, or unsubstituted C₂-C₁₀ alkynyl;
R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;
R¹³ is hydrogen, unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl; and,
R¹⁷ is hydrogen or methyl.

17. The compound as in claim 16 of the structure 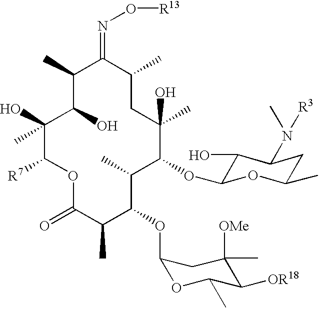
wherein

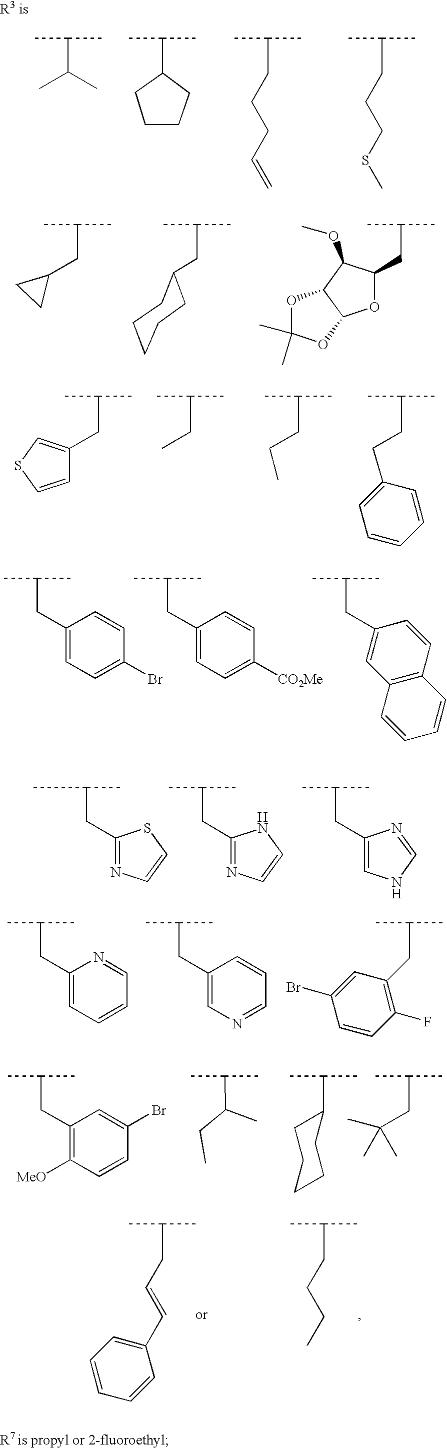

18. A method of treating a subject suffering from impaired GI motility comprising:
administering a composition comprising a compound of the formula 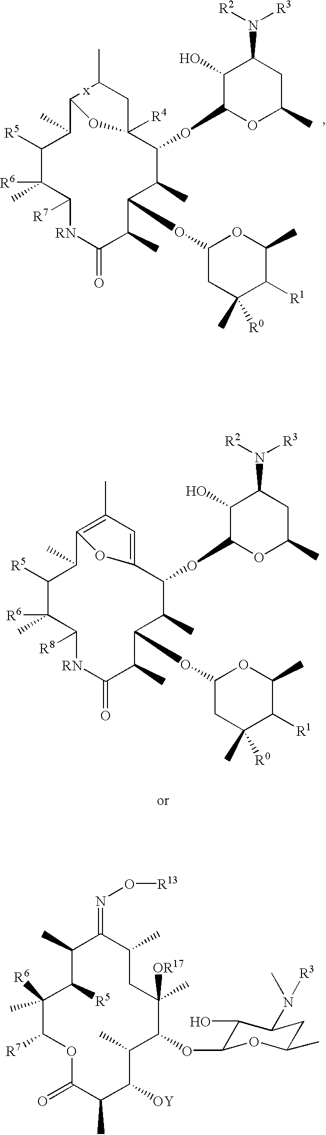
wherein:
R is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;
R⁰ is hydroxyl or methoxy;
R¹ is selected from the group consisting of hydrogen, hydroxyl, halide, NH₂, OR⁹, 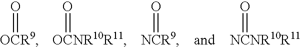
where
R⁹ substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl, and R¹⁰ and R¹¹ are each independently hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl;
R² and R³ are each independently selected from the group consisting of hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, or unsubstituted alkynylaryl, or R² and R³ together form a cycloalkyl or an aryl moiety;
R⁴ is hydrogen or methyl;
R⁵ is hydroxyl or oxo;
R⁶ is hydrogen, hydroxyl, or OR¹² where R¹² is substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, or unsubstituted C₂-C₁₀ alkynyl;
R⁷ is methyl, unsubstituted C₃-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;
R⁸ is unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;
R¹³ is hydrogen, unsubstituted C₁-C₁₀ alkyl, substituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted alkylaryl, unsubstituted alkylaryl, substituted alkenylaryl, unsubstituted alkenylaryl, substituted alkynylaryl, or unsubstituted alkynylaryl;
R¹⁷ is hydrogen or methyl;
x is a single or a double bond; and,
Y is hydrogen, substituted C₁-C₁₀ alkyl, unsubstituted C₁-C₁₀ alkyl, substituted C₂-C₁₀ alkenyl, unsubstituted C₂-C₁₀ alkenyl, substituted C₂-C₁₀ alkynyl, unsubstituted C₂-C₁₀ alkynyl, substituted aryl, unsubstituted aryl, substituted alkylaryl, unsubstituted alkylaryl, substituted alkenylaryl, unsubstituted alkenylaryl, substituted alkynylaryl, unsubstituted alkynylaryl, unsubstituted cladinose, or substituted cladinose.

19. The method as in claim 18 wherein the subject is a human suffering from gastroparesis, gastroesophageal reflux disease, anorexia, gall bladder stasis, postoperative paralytic ileus, scleroderma, intestinal pseudoobstruction, gastritis, emesis, and chronic constipation (colonic inertia).
